(19) United States
(12) Patent Application Publication
(10) Pub. No.: US 2026/0103443 A1
(43) Pub. Date: Apr. 16, 2026

(54) CHEMICAL COMPOUND

(71) Applicant: LMS Co., Ltd., Pyeongtaek-si (KR)

(72) Inventors: Seung Chul LEE, Pyeongtaek-si (KR); Hee Kyeong KIM, Pyeongtaek-si (KR); Joon Ho JUNG, Pyeongtaek-si (KR)

(21) Appl. No.: 19/333,250

(22) Filed: Sep. 18, 2025

(30) Foreign Application Priority Data
Oct. 11, 2024 (KR) 10-2024-0138184

(51) Int. Cl. C07D 209/60; C07B 59/00; G02B 5/22

(52) U.S. Cl. CPC C07D 209/60; C07B 59/002; G02B 5/223; C07B 2200/05

(57) ABSTRACT

Provided is a compound and its use. Provided is a chemical compound having excellent heat resistance, oxidation resistance, and stably maintaining light absorption characteristics even when maintained under high temperature conditions or high temperature and high humidity conditions. Provided is also a resin film having desired light characteristics secured by applying the chemical compound. Provided are also applications of the chemical compound.

### CROSS-REFERENCE TO RELATED APPLICATION(S)

This application is based on and claims priority under 35 U.S.C. 119 to Korean Patent Application No. 10-2024-0138184, filed on Oct. 11, 2024, in the Korean Intellectual Property Office, the disclosure of which is herein incorporated by reference in its entirety.

### TECHNICAL FIELD

The present specification relates to a chemical compound and its applications.

### BACKGROUND

Chemical compounds that can be used as absorbents, for example, the chemical compounds that can transmit light in the visible range with high transmittance while absorbing light in the near-infrared range, can be applied to various purposes. For example, in image capturing devices utilizing CCD (Charge-Coupled Device) or CMOS (Complementary Metal-Oxide-Semiconductor) as image sensors or infrared sensors, an absorbent can be used because they include silicon photodiodes that are sensitive to the near-infrared region.

Known absorbents may include phthalocyanine-based compounds, cyanine-based compounds, metal dithiol complex-based compounds, squarylium-based compounds, and diimonium salt-based compounds. Phthalocyanine compounds are known as near-infrared compounds, but they have a problem of high absorption in the visible light range. In addition, the cyanine-based compounds have a narrow near-infrared absorption range that can be absorbed by a single compound, so they have to be mixed with other compounds for use. In addition, the metal dithiol complex compounds have a problem that they have low solubility, so additional dispersion equipment is required when applied to films. Furthermore, they are difficult to apply to applications that require high transmittance.

In addition, iminium or diimonium compounds are known as compounds capable of absorbing light having a wavelength of 900 nm or more. However, iminium or diimonium compounds have a problem in that they lose their absorption properties in high temperature and/or high humidity environments due to low thermal stability and low resistance. A composition containing the chemical compound has a problem in that it loses its spectral properties when cured due to low thermal stability and low oxidation resistance. There is a need for chemical compounds that possess high thermal stability while maintaining light absorption properties in high-temperature and/or high-humidity environments.

### SUMMARY

The objective of the present specification is to disclose a chemical compound and its applications.

The objective of the present specification is to disclose a chemical compound having excellent heat resistance and oxidation resistance where it can stably maintain light absorption characteristics even when high temperature conditions or high temperature and high humidity conditions are maintained.

The objective of the specification is to provide a resin film having desired light characteristics secured by applying the chemical compound.

According to an embodiment of the present invention, there is provided that a chemical compound comprises deuterium and represented by Chemical Formula 1:

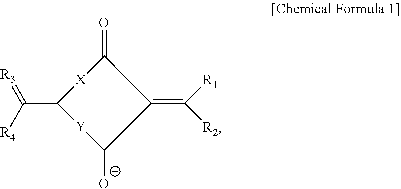

where, in Chemical Formula 1, one of X and Y is a single bond and the other is a double bond; R₁ forms a structure represented by Chemical Formula 2; R₂ is hydrogen or deuterium or R₁ and R₂ together form a structure represented by Chemical Formula 3; R₃ forms a structure represented by Chemical Formula 4; and R₄ is hydrogen or deuterium or R₃ and R₄ together form a structure represented by Chemical Formula 5; where, in Chemical Formula 2:

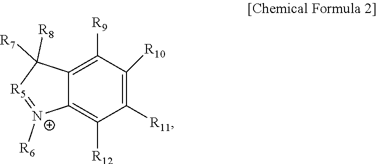

R₅ is carbon as R₁ of Chemical Formula 1; R₆ to R₈ are each independently hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, or a cyano group; and R₉ to R₁₂ are each independently hydrogen or deuterium wherein R₉ and R₁₀, R₁₀ and R₁₁, or R₁₁ and R₁₂ are linked to each other to form an aromatic structure which is unsubstituted or substituted with deuterium; where, in Chemical Formula 3:

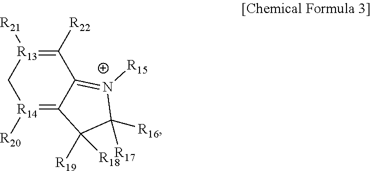

one of R₁₃ and R₁₄ is R₁ of Chemical Formula 1 and the other is R₂ of Chemical Formula 1 where each of R₁₃ and R₁₄ is carbon; and R₁₅ to R₂₀ are each independently hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, a cyano group, or —NR^{a}R^{b} where R^{a} and R^{b} are each independently hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group or a cyano group; and R₂₁ and R₂₂ are each independently hydrogen or deuterium, or are linked to each other to form an aromatic structure which is unsubstituted or substituted with deuterium; where, in Chemical Formula 4:

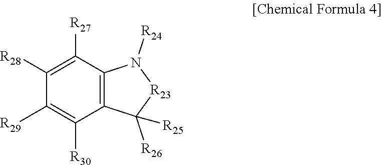

R₂₃ is carbon as R₃ of Chemical Formula 1 and R₂₄ to R₂₆ are each independently hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, or a cyano group; and R₂₇ to R₃₀ are each independently hydrogen or deuterium, and R₂₇ and R₂₈, R₂₈ and R₂₉, or R₂₉ and R₃₀ are linked to each other to form a benzene structure which is unsubstituted or substituted with deuterium; and where, in Chemical Formula 5:

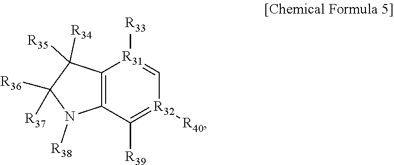

one of R₃₁ and R₃₂ is R₃ of Chemical Formula 1, and the other is R₄ of Chemical Formula 1 where each of R₃₁ and R₃₂ is carbon; and R₃₃ to R₃₈ are each independently hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, a cyano group, or —NR^{a}R^{b} where R^{a} and R^{b} are each independently hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, or a cyano group; and R₃₉ and R₄₀ are each independently be hydrogen or deuterium or are linked to each other to form a benzene structure which is unsubstituted or substituted with deuterium.

In an embodiment, R₁ forms a structure of Chemical Formula 2 and R₂ is deuterium for the chemical compound.

In an embodiment, R₆ to R₈ are each independently hydrogen, deuterium, an alkyl group, an alkyloxy group, or an alkoxy group; and R₉ to R₁₂ are each independently deuterium in Chemical Formula 2 for the chemical compound.

In an embodiment, R₆ to R₈ are each independently hydrogen, deuterium, an alkyl group, an alkyloxy group, or an alkoxy group; R₉ and R₁₀ are connected to each other to form an aromatic structure substituted with deuterium; and R₁₁ and R₁₂ are deuterium in Chemical Formula 2 for the chemical compound.

In an embodiment, R₃ forms a structure of Chemical Formula 4 and R₄ is deuterium in Chemical Formula 1 for the chemical compound.

In an embodiment, R₂₄ to R₂₆ are each independently hydrogen, deuterium, an alkyl group, an alkyloxy group, or an alkoxy group; and R₂₇ to R₃₀ are each deuterium in Chemical Formula 4 for the chemical compound.

In an embodiment, R₂₄ to R₂₆ are each independently hydrogen, deuterium, an alkyl group, an alkyloxy group, or an alkoxy group; R₂₇ and R₂₈ are each deuterium; and R₂₉ and R₃₀ are connected to each other to form an aromatic structure substituted with deuterium in Chemical Formula 4 for the chemical compound.

In an embodiment, R₁ and R₂ of Chemical Formula 1 form the structure of Chemical Formula 3 in Chemical Formula 1 for the chemical compound.

In an embodiment, R₁₅ to R₂₀ are each independently hydrogen, deuterium, an alkyl group, an alkyloxy group, an alkoxy group, or —NR^{a}R^{b} where R^{a} and R^{b} are each independently hydrogen, deuterium, an alkyl group, an alkyloxy group, or an alkylcarbonyl group; and R₂₁ and R₂₂ are each deuterium in Chemical Formula 3 for the chemical compound.

In an embodiment, R₁₅ to R₂₀ are each independently hydrogen, deuterium, an alkyl group, an alkyloxy group or an alkoxy group; and R₂₁ and R₂₂ are connected to each other to form an aromatic structure substituted with deuterium in Chemical Formula 3 for the chemical compound.

In an embodiment, R₃ and R₄ form the structure of Chemical Formula 5 in Chemical Formula 1 for the chemical compound.

In an embodiment, R₃₃ to R₃₈ are each independently hydrogen, deuterium, an alkyl group, an alkyloxy group, an alkoxy group, or —NR^{a}R^{b} wherein Rₐ and R_{b} are each independently hydrogen, deuterium, an alkyl group, an alkyloxy group, or an alkylcarbonyl group; and R₃₉ and R₄₀ are each deuterium in Chemical Formula 5 for the chemical compound.

In an embodiment, R₃₃ to R₃₈ are each independently hydrogen, deuterium, an alkyl group, an alkyloxy group, or an alkoxy group; and R₃₉ and R₄₀ are connected to each other to form an aromatic structure substituted with deuterium in Chemical Formula 5 for the chemical compound.

In an embodiment, a chemical compound of Chemical Formula 1 is represented by any one of Chemical Formulas 6 to 9:

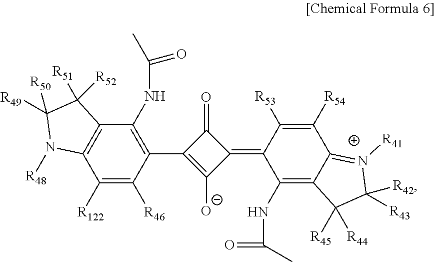

where, in Chemical Formula 6, R₄₁ to R₅₄ are each independently hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, a cyano group, or —NR^{a}R^{b} where R^{a} and R^{b} are each independently hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, or a cyano group; in Chemical Formula 7:

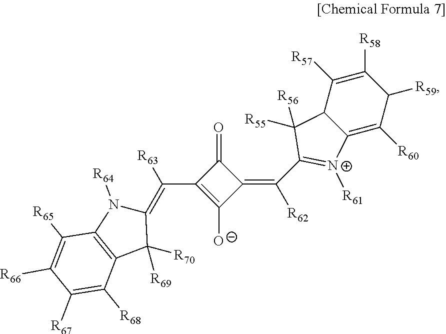

R₅₅ to R₇₀ are each independently hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, a cyano group, or —NR^{a}R^{b} where R^{a} and R^{b} are each independently hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, or a cyano group; in Chemical Formula 8:

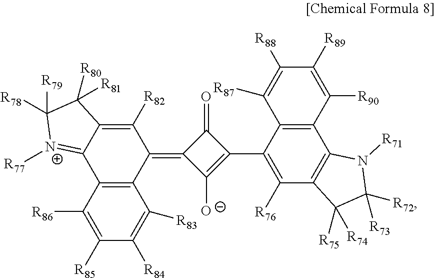

R₇₁ to R₉₀ are each independently hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, a cyano group, or —NR^{a}R^{b} where R^{a} and R^{b} are each independently hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, or a cyano group; and in Chemical Formula 9:

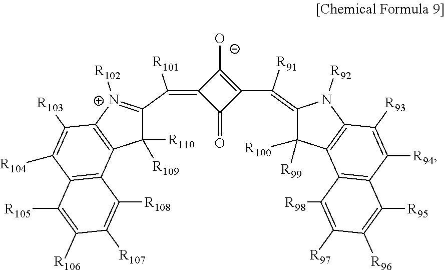

R₉₁ to R₁₁₀ are each independently hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, a cyano group, or —NR^{a}R^{b} where R^{a} and R^{b} are each independently hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, or a cyano group for the chemical compound.

In an embodiment, a deuterium substitution rate is 10% or higher for the chemical compound.

According to another embodiment of the present invention, there is provided that a composition comprises a resin component and the chemical compound where the composition contains 0.001 to 10 parts by weight of the chemical compound with respect to 100 parts by weight of the resin component.

According to another embodiment of the present invention, there is provided that a resin film comprises the resin component; and the chemical compound where the resin film exhibits an absorption maximum within a wavelength range of 650 nm to 900 nm.

In another embodiment, ΔA of Equation 1 for the resin film is 50% or less:

$$△A = 100 \times ( {A}_{f} - {A}_{i} ) / {A}_{i} , \qquad [ Equation 1 ]$$

where A_{f} is a transmittance at a maximum absorption wavelength of the resin film maintained at 85° C. and a relative humidity of 85% for 120 hours and Aᵢ is a transmittance at a maximum absorption wavelength of the resin film before being maintained at 85° C. and a relative humidity of 85% for 120 hours for the resin film.

In another embodiment, an absolute value of Δλ of Equation 2 for the resin film is 10% or less:

$$△λ = 100 \times ( {λ}_{f} - {λ}_{i} ) / {λ}_{i} , \qquad [ Equation 2 ]$$

where λ_{f} is a maximum absorption wavelength of the resin film maintained at 85° C. and a relative humidity of 85% for 120 hours and Aᵢ a maximum absorption wavelength of the resin film before being maintained at 85° C. and a relative humidity of 85% for 120 hours for the resin film.

According to yet another embodiment of the present invention, there is provided that an optical filter comprises that a substrate layer; and the resin film formed on one or both sides of the substrate layer.

### BRIEF DESCRIPTION OF THE DRAWINGS

FIGS. 1 to 3 are exemplary structures of optical filters disclosed in the present specification.

FIG. 4 to 11 are the absorption characteristics of resin films including chemical compounds of Embodiments or Comparative Examples before and after high temperature and high humidity test.

### DETAILED DESCRIPTION

The term “room temperature” refers to the natural temperature, which is not artificially heated or cooled. For example, room temperature may be any temperature within the range of about 10° C. to 30° C., or about 23° C., about 25° C., or about 27° C. Unless otherwise specified, properties affected by measurement temperature are properties measured at room temperature. The unit of temperature as mentioned is Celsius (° C.) unless otherwise specified.

The term “atmospheric pressure refers to the natural pressure which is not artificially pressurized or depressurized. It typically refers to a pressure of about 730 mmHg to 790 mmHg. Unless otherwise specified, properties affected by measurement pressure are properties measured at atmospheric pressure.

Among the physical properties mentioned in this specification, properties affected by measured humidity are properties measured at standard humidity unless otherwise specified. Standard humidity refers to a relative humidity within the range of 40% to 60%, for example, approximately 55% or approximately 60%.

If an optical property (e.g., refractive index) mentioned in this specification is a wavelength-dependent property, unless otherwise specified, the optical property is a property for light with a wavelength of 520 nm.

The terms “transmittance” and “absorptivity” refer to the actual transmittance (actual transmittance) or actual absorption (actual absorption) confirmed within a specific wavelength or a predetermined wavelength range unless otherwise specified and are the transmittance or absorption based on an incident angle of 0 degrees. The term “average transmittance” or “average absorption” refers to the arithmetic mean of the transmittance or absorption measured at each wavelength, increasing the wavelength by 1 nm from the shortest wavelength within a given wavelength range unless otherwise specified. For example, the average transmittance or average absorption within the wavelength range of 350 nm to 360 nm refers to the arithmetic mean of the transmittance or absorption measured at wavelengths of 350 nm, 351 nm, 352 nm, 353 nm, 354 nm, 355 nm, 356 nm, 357 nm, 358 nm, 359 nm, and 360 nm.

The term “maximum transmittance” or “maximum absorption” refers to the maximum transmittance or maximum absorption measured at each wavelength, increasing the wavelength by 1 nm from the shortest wavelength within a given wavelength range. For example, the maximum transmittance or maximum absorption within the wavelength range of 350 nm to 360 nm is the highest transmittance or absorption among the transmittances measured at wavelengths of 350 nm, 351 nm, 352 nm, 353 nm, 354 nm, 355 nm, 356 nm, 357 nm, 358 nm, 359 nm, and 360 nm.

The term “minimum transmittance” or “minimum absorption” refers to the minimum transmittance or minimum absorption when the transmittance or absorption is measured at each wavelength while increasing the wavelength by 1 nm from the shortest wavelength within a given wavelength range. For example, the minimum transmittance or minimum absorptivity within the wavelength range of 350 nm to 360 nm is the lowest transmittance or absorptivity among the transmittances or absorptivity measured at wavelengths of 350 nm, 351 nm, 352 nm, 353 nm, 354 nm, 355 nm, 356 nm, 357 nm, 358 nm, 359 nm, and 360 nm.

In the present specification, the angle of incidence is an angle based on the normal to the surface to be evaluated. For example, the transmittance of an optical filter at an incident angle of 0 degrees refers to the transmittance for light incident in a direction substantially parallel to the normal to the surface of the optical filter. This definition of the angle of incidence also applies equally to other characteristics, such as transmittance or absorptivity.

The term “alkyl group” may, unless otherwise specified, refer to a straight-chain or branched alkyl group having 1 to 30 carbon atoms, 1 to 24 carbon atoms, 1 to 20 carbon atoms, 1 to 16 carbon atoms, 1 to 12 carbon atoms, 1 to 8 carbon atoms, or 1 to 4 carbon atoms; or a cyclic alkyl group having 3 to 30 carbon atoms, 3 to 24 carbon atoms, 3 to 20 carbon atoms, 3 to 16 carbon atoms, 3 to 12 carbon atoms, or 3 to 8 carbon atoms. The cyclic alkyl group includes an alkyl group consisting solely of a ring structure and an alkyl group including a ring structure.

The term “alkenyl group” may, unless otherwise specified, refer to a straight-chain or branched acyclic alkenyl group having 2 to 30 carbon atoms, 2 to 24 carbon atoms, 2 to 20 carbon atoms, 2 to 16 carbon atoms, 2 to 12 carbon atoms, 2 to 8 carbon atoms, or 2 to 4 carbon atoms; or a cyclic alkenyl group having 3 to 30 carbon atoms, 3 to 24 carbon atoms, 3 to 20 carbon atoms, 3 to 16 carbon atoms, 3 to 12 carbon atoms, or 3 to 8 carbon atoms. The cyclic alkenyl group also includes an alkenyl group consisting solely of a ring structure and an alkenyl group comprising a ring structure.

The term “alkynyl group” may refer to a straight-chain or branched acyclic alkynyl group having 2 to 20 carbon atoms, 2 to 16 carbon atoms, 2 to 12 carbon atoms, 2 to 8 carbon atoms, or 2 to 4 carbon atoms; or a cyclic alkynyl group having 3 to 30 carbon atoms, 3 to 24 carbon atoms, 3 to 20 carbon atoms, 3 to 16 carbon atoms, 3 to 12 carbon atoms, or 3 to 8 carbon atoms. The cyclic alkynyl group also includes alkynyl groups that exist solely in a ring structure and alkynyl groups that include a ring structure.

Unless otherwise specified, the term “alkoxy group” may refer to a straight-chain or branched alkoxy group having 1 to 30 carbon atoms, 1 to 24 carbon atoms, 1 to 20 carbon atoms, 1 to 16 carbon atoms, 1 to 12 carbon atoms, 1 to 8 carbon atoms, or 1 to 4 carbon atoms; or a cyclic alkoxy group having 3 to 30 carbon atoms, 3 to 24 carbon atoms, 3 to 20 carbon atoms, 3 to 16 carbon atoms, 3 to 12 carbon atoms, or 3 to 8 carbon atoms. The cyclic alkoxy group includes alkoxy groups that exist solely in a ring structure and alkoxy groups that include a ring structure.

The term “aryl group” refers to a substituent formed by the removal of a hydrogen atom from an aromatic hydrocarbon ring compound, which may be a monocyclic or polycyclic ring compound. Unless otherwise specified, the aryl group may be an aryl group having 6 to 48 carbon atoms, 6 to 42 carbon atoms, 6 to 36 carbon atoms, 6 to 30 carbon atoms, 6 to 24 carbon atoms, 6 to 18 carbon atoms, or 6 to 12 carbon atoms, such as a phenyl group, a tolyl group, a xylene group, or a naphthyl group. The aryl group may be, for example, a heteroaryl group, and the heteroaryl group is a structure in which the ring structure of the aryl group includes a heteroatom other than a carbon atom, such as O, N, or S.

The alkyl group, alkenyl group, alkynyl group, alkoxy group, and aryl group may be substituted with any one or more substituents. In this case, the substituent may be at least one selected from the group consisting of halogen (chlorine (Cl), iodine (I), bromine (Br), or fluorine (F)), an aryl group, a heteroaryl group, an epoxy group, an alkoxy group, a cyano group, an amino group, a carboxyl group, an acryloyl group, a methacryloyl group, an acryloyloxy group, a methacryloyloxy group, a carbonyl group, and a hydroxy group, but it is not limited to.

The present specification discloses a chemical compound. The chemical compound may be an absorbent. The term “absorbent” means a chemical compound capable of absorbing light of any wavelength range.

The chemical compound may be a compound represented by Chemical Formula 1:

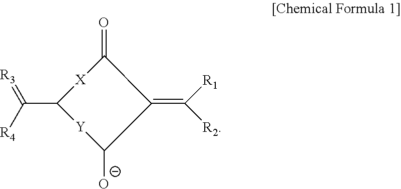

In Chemical Formula 1, either X or Y may be a single bond and the other may be a double bond. In one example, X in Chemical Formula 1 may be a single bond and Y may be a double bond.

In Chemical Formula 1, either R₁ or R₂ (e.g., R₁) can form a structure of Chemical Formula 2 and the other (e.g., R₂) can be hydrogen or deuterium. In the above case, for example, the substituent that does not form the structure of Chemical Formula 2 can be deuterium:

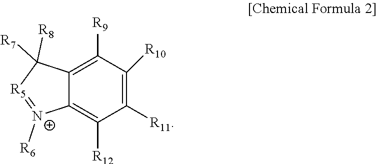

In Chemical Formula 2, R₅ can be carbon as R₁ of Chemical Formula 1. In Chemical Formula 2, R₆ to R₈ can each independently be hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, or a cyano group.

In Chemical Formula 2, R₉ to R₁₂ can each independently be hydrogen or deuterium. In some cases, R₉ and R₁₀, R₁₀ and R₁₁, and/or R₁₁ and R₁₂ in Chemical Formula 2 may be linked to each other to form an aromatic structure. In the above case, the substituent that does not form an aromatic structure may be hydrogen or deuterium.

The above aromatic structure may be, for example, an aromatic structure having 6 to 36, 6 to 30, 6 to 24, 6 to 18, or 6 to 12 carbon atoms and may be, for example, a benzene structure. These aromatic structures or benzene structures may be substituted with at least one deuterium atom.

In another example, R₁ and R₂ of Chemical Formula 1 can together form the structure of Chemical Formula 3 below:

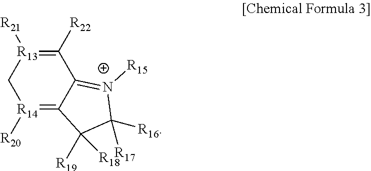

In Chemical Formula 3, either R₁₃ or R₁₄ may be R₁ of Chemical Formula 1 and the other may be R₂ of Chemical Formula 2. Each of R₁₃ and R₁₄ may be carbon.

In Chemical Formula 3, R₁₅ to R₂₀ may each independently be hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, a cyano group, or —NR^{a}R^{b}. In this case, R^{a} and R^{b} may each independently be hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, or a cyano group.

In Chemical Formula 3, R₂₁ and R₂₂ can each independently be hydrogen or deuterium. In another example, R₂₁ and R₂₂ can be linked to each other to form an aromatic structure. The aromatic structure can be, for example, an aromatic structure having 6 to 36, 6 to 30, 6 to 24, 6 to 18, or 6 to 12 carbon atoms, and can be, for example, a benzene structure. The aromatic structure or the benzene structure can be substituted with at least one deuterium.

Additionally, in Chemical Formula 1, either one of R₃ and R₄ (e.g., R₃) may form a structure of Chemical Formula 4 below and the other (e.g., R₄) may be hydrogen or deuterium. In this case, the substituent that does not form a structure of Chemical Formula 4 may be, for example, deuterium:

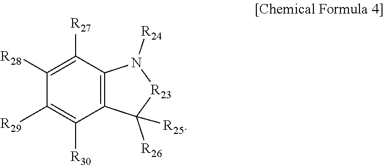

In Chemical Formula 4, R₂₃ can be carbon as R₃ in Chemical Formula 1. R₂₄ in Chemical Formula 4 and R₂₆ can each independently be hydrogen, deuterium, an alkyl group, an alkyloxy group, an alkylcarbon group, an alkoxy group, an alkylamino group, an alkylsilyl group, an amino group, a nitro group, a nitrile group, a hydroxy group, or a cyano group.

In Chemical Formula 4, R₂₇ to R₃₀ may each independently be hydrogen or deuterium. In this case, R₂₇ and R₂₈, R₂₈ and R₂₉, and/or R₂₉ and R₃₀ may be linked to each other to form an aromatic structure. In the above case, the substituent that does not form an aromatic structure may be hydrogen or deuterium.

The above aromatic structure may be, for example, an aromatic structure having 6 to 36, 6 to 30, 6 to 24, 6 to 18, or 6 to 12 carbon atoms, and may be, for example, a benzene structure. These aromatic structures or benzene structures may be substituted with at least one deuterium atom.

In another example, R₃ and R₄ in Chemical Formula 1 can together form the structure of formula 5:

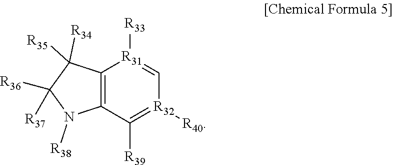

In Chemical Formula 5, either one of R₃₁ and R₃₂ may be R₃ of Chemical Formula 1 and the other may be R₄ of Chemical Formula 1. Each of R₃₁ and R₃₂ may be carbon.

In Chemical Formula 5, R₃₃ to R₃₈ can each independently be hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, a cyano group, or —NR^{a}R^{b}. In the above, R^{a} and R^{b} can each independently be hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, or a cyano group.

In Chemical Formula 5, R₃₉ and R₄₀ may each independently be hydrogen or deuterium, or may be linked to each other to form an aromatic structure. The aromatic structure may be, for example, an aromatic structure having 6 to 36, 6 to 30, 6 to 24, 6 to 18, or 6 to 12 carbon atoms and may be, for example, a benzene structure. The aromatic structure or benzene structure may be substituted with at least one deuterium.

The chemical compound of Chemical Formula 1 may be, for example, a chemical compound represented by any one of Chemical Formulas 6 to 9:

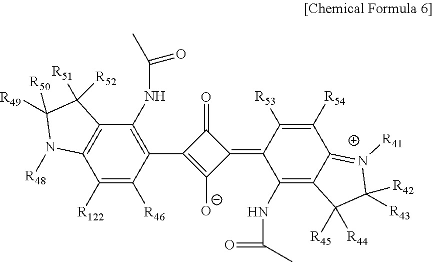

In Chemical Formula 6, R₄₁ to R₅₄ may each independently be hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, a cyano group, or —NR^{a}R^{b}. In this case, R^{a} and R^{b} may each independently be hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, or a cyano group. In one example, in Chemical Formula 6, R₄₁ to R₄₅ and R₄₈ to R₅₂ are each independently hydrogen, an alkyl group, an alkyloxy group, or an alkylcarbonyl group and R₄₆, R₄₇, R₅₃ and R₅₄ can each be deuterium.

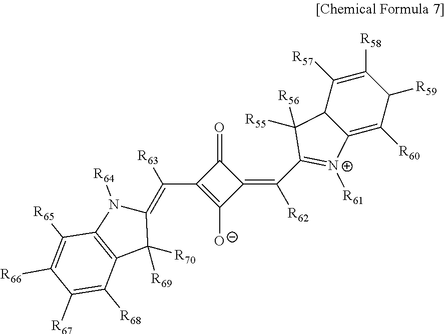

In Chemical Formula 7, R₅₅ to R₇₀ may each independently be hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, a cyano group, or —NR^{a}R^{b}. In this case, R^{a} and R^{b} may each independently be hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, or a cyano group.

In one example, in Chemical Formula 7, R₅₅, R₅₆, R₆₁, R₆₄, R₆₉ and R₇₀ are each independently hydrogen, an alkyl group, an alkyloxy group, or an alkylcarbonyl group and R₅₇ to R₆₀, R₆₂, R₆₃, R₆₅ to R₆₈ can each be deuterium.

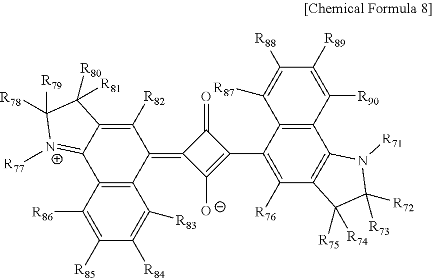

In Chemical Formula 8, R₇₁ to R₉₀ can each independently be hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, a cyano group, or —NR^{a}R^{b}. In this case, R^{a} and R^{b} can each independently be hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, or a cyano group.

In one example, in Chemical Formula 8, R₇₁ to R₇₅ and R₇₇ to R₈₁ may each independently be hydrogen, an alkyl group, an alkyloxy group, or an alkylcarbonyl group and R₇₆ and R₈₂ to R₉₀ may each be deuterium.

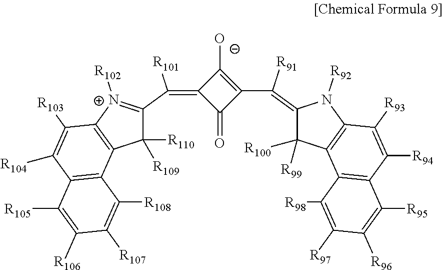

In Chemical Formula 9, R₉₁ to R₁₁₀ may each independently be hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, a cyano group, or —NR^{a}R^{b}. In this case, R^{a} and R^{b} may each independently be hydrogen, deuterium, an alkyl group, an alkyloxy group, an alkylcarbonyl group, an alkoxy group, an alkylamino group, an alkylsilyl group, an amino group, a nitro group, a nitrile group, a hydroxy group, or a cyano group. In one example, in Chemical Formula 9, R₉₂, R₉₉, R₁₀₀, R₁₀₂, R₁₀₉, and R₁₁₀ are each independently hydrogen, an alkyl group, an alkyloxy group, or an alkylcarbonyl group and R₉₁, R₉₃ to R₉₈, R₁₀₁, and R₁₀₃ to R₁₀₈ can each be deuterium.

The chemical compounds represented by Chemical Formulas 1 and 6 to 9 contain an absorption group, and thus, can have a structure capable of exhibiting the characteristic of absorbing light of a desired wavelength. For example, the absorption edge may be a frame or structure with a so-called resonance structure and/or conjugated bond. Light absorption by chemical compounds, particularly organic compounds, is known to be caused by the energy difference (ΔE) between the ground state and the excited state. Such difference can also be explained by the energy difference between the HOMO (Highest Unoccupied Molecular Orbital) and LUMO (Lowest Unoccupied Molecular Orbital).

Generally, organic absorbents may include resonance structures and/or conjugated bonds as absorption edges capable of exhibiting light absorption effects. Accordingly, the chemical compound may form a frame that enables it to exhibit the desired light absorption characteristics as a whole.

The specific types of the absorption edge or frame described above are not particularly limited. As is well known, the resonance effect refers to the interaction between a molecule's isolated pair of electrons and an adjacent π-bonded electron pair. The substituents or frames that induce this resonance effect are well known. In addition, it is known that a conjugated bond is a system where two or more double bonds are formed with one single bond in between, and as the length of this conjugated bond increases, the energy difference between the HOMO and LUMO decreases thereby causing the absorption band to shift toward longer wavelengths.

For example, the absorption edge may be a frame or structure that allows the chemical compound to exhibit an absorption maximum within a wavelength range of 650 nm to 900 nm. The chemical compound may exhibit an absorption maximum wavelength within a range of 650 nm to 900 nm. The lower limit of the absorption maximum wavelength may be, in other examples, about 650 nm, 660 nm, 670 nm, 680 nm, 690 nm, 700 nm, 710 nm, 720 nm, 730 nm, 740 nm, 750 nm, 760 nm, or 765 nm. In addition, the upper limit of the absorption maximum wavelength may be about 900 nm, 800 nm, 790 nm, 780 nm, 770 nm, 760 nm, 750 nm, 740 nm, 730 nm, 720 nm, or 710 nm. The absorption maximum wavelength may be within a range that is equal to or greater than any one of the lower limits described above; or within a range that is equal to or greater than any one of the lower limits described above and at the same time less than or equal to any one of the upper limits described above. As described above, the resonance structure and conjugated bond determine the energy difference (ΔE) between the ground state and the excited state of the compound or the energy difference between the HOMO (Highest Unoccupied Molecular Orbital) and the LUMO (Lowest Unoccupied Molecular Orbital). Since the absorption maximum wavelength is determined by this energy difference, the structure of the absorption edge can be determined so that the chemical compound can have the absorption maximum wavelength in the range described above.

At least one or two or more or all of R₄₆, R₄₇, R₅₃, and R₅₄ in Chemical Formula 6; at least one or two or more or all of R₅₇ to R₆₀, R₆₂, R₆₃, and R₆₅ to R₆₈ in Chemical Formula 7; at least one or two or more or all of R₇₆ and R₈₂ to R₉₀ in Chemical Formula 8; and/or at least one or two or more or all of R₉₁, R₉₃ to R₉₈, R₁₀₁, and R₁₀₃ to R₁₀₈ in Chemical formula 9 may include deuterium. Here, the meaning of including deuterium can include, for example, the case where the substituent is deuterium, or an alkyl group substituted with deuterium, an alkenyl group substituted with at least one deuterium, an alkynyl group substituted with at least one deuterium, an alkoxy group substituted with at least one deuterium, an aryl group substituted with at least one deuterium, an aryloxy group substituted with at least one deuterium, an arylamino group substituted with at least one deuterium, an alkylamino group substituted with at least one deuterium, a heteroaryl group substituted with at least one deuterium, an alkylsilyl group substituted with at least one deuterium, or an arylsilyl group substituted with at least one deuterium.

The chemical compound disclosed in this specification can secure excellent heat resistance and/or oxidation resistance without affecting the absorption characteristics of the compound by deuterium. More specifically, the chemical compound represented by Chemical Formula 1 can secure excellent heat resistance and/or oxidation resistance by deuterium included and the chemical compound represented by any one of Chemical Formulas 6 to 9 can secure excellent heat resistance and/or oxidation resistance by deuterium included.

The lower limit of the number of deuterium atoms contained in the chemical compound may be 1, 2, 3, 4, 5, 6, 7, 8, 9, 10, 11, 12, 13, or 14 and the upper limit may be 200, 100, 90, 80, 70, 60, 50, 40, 30, 20, 19, 18, 17, 16, 15, 10, 9, 8, 7, 6, 5 or 4. The number of deuterium atoms may be within a range that is equal to or greater than any one of the lower limits described above or may be within a range that is equal to or greater than any one of the lower limits described above and at the same time less than or equal to any one of the upper limits described above. The number of deuterium atoms above may be the number of moles of deuterium atoms contained in one mole of the chemical compound.

The deuterium substitution rate of the chemical compound may be at a certain level or higher. The deuterium substitution rate is theoretically the ratio of the number of moles of deuterium after deuterium substitution based on the number of moles of all hydrogens that one mole of the chemical compound has before deuterium substitution. The lower limit of the deuterium substitution rate can be about 10%, 15%, 20%, 25%, 30%, 40%, 45%, 50%, 55%, 60%, 65%, 70%, 75%, 80%, 85%, or 90% and the upper limit can be about 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, or 75%. The above ratio may be within a range that is equal to or greater than any one of the lower limits described above or within a range that is equal to or greater than any one of the lower limits described above and at the same time less than or equal to any one of the upper limits described above. In addition, the deuterium substitution rate may mean the degree where hydrogen directly bonded to carbon is substituted with deuterium. The method of measuring the deuterium substitution rate is described below in “5. Deuterium Substitution Rate” in the present specification.

The chemical compound disclosed in the present specification can secure heat resistance by controlling the deuterium substitution rate. The bond between carbon and deuterium has lower stretching and bending energy than the bond between carbon and hydrogen. Therefore, it is understood that the bond between carbon and deuterium can reduce the vibration energy within the molecule compared to the bond between carbon and hydrogen, and thus, the chemical compound may maintain the absorption characteristics and secure heat resistance even in high temperature and high humidity environments.

The chemical compound in the present specification can also secure oxidation resistance by controlling the deuterium substitution rate. In the case of a deuterium-substituted chemical compound, as mentioned above, the intramolecular vibration energy is reduced, and as a result, the oxidation potential is increased. It is understood that, due to the increase in the oxidation potential, oxidation is suppressed and the absorption characteristics can also be maintained even in a high temperature and high humidity environment compared to a non-deuterium-substituted compound.

The chemical compound can have an appropriate level of molar weight. For example, the lower limit of the molar weight may be about 300 g/mol, 400 g/mol, 450 g/mol, 500 g/mol, 505 g/mol, 510 g/mol, 515 g/mol, 520 g/mol, 525 g/mol, 530 g/mol, 535 g/mol, 540 g/mol, 545 g/mol, 550 g/mol, 555 g/mol, 560 g/mol, 565 g/mol, 570 g/mol, 575 g/mol, 580 g/mol, 585 g/mol, 590 g/mol, 595 g/mol, 600 g/mol, 605 g/mol, 610 g/mol, 615 g/mol, 620 g/mol, or 625 g/mol. The upper limit of the molar weight may be about 3,000 g/mol, 2,500 g/mol, 2,000 g/mol, 1,500 g/mol, 1,000 g/mol, 900 g/mol, 800 g/mol, 700 g/mol, 690 g/mol, 680 g/mol, 670 g/mol, 660 g/mol, 650 g/mol, 640 g/mol, 630 g/mol, 620 g/mol, 610 g/mol, 600 g/mol, 550 g/mol, 540 g/mol, 530 g/mol, 520 g/mol, or 510 g/mol. The molar weight may be within a range that is equal to or greater than any one of the lower limits described above; within a range that is less than or equal to any one of the upper limits described above; or within a range that is equal to or greater than any one of the lower limits described above and at the same time less than or equal to any one of the upper limits described above.

The chemical compound may have excellent heat resistance. For example, the 5% thermal decomposition temperature (“Td 5%”) of the chemical compound may be within a predetermined range. For example, the lower limit of Td 5% of the chemical compound may be about 200° C., 210° C., 220° C., 230° C., 240° C., 250° C., 260° C., 270° C., 280° C., or 290° C. The upper limit of Td 5% may be about 500° C., 480° C., 460° C., 440° C., 420° C., 400° C., 380° C., 360° C., 350° C., 340° C., 330° C., 320° C., 310° C., 300° C., 290° C., 280° C., 270° C., 260° C., or 250° C. Td 5% may be within a range that is equal to or greater than any one of the above-described lower limits; within a range that is less than or equal to any one of the above-described upper limits; or within a range that is equal to or greater than any one of the above-described lower limits and less than or equal to any one of the above-described upper limits.

Td 5% is the temperature where 95% weight loss occurs in TGA (Thermogravimetric analysis) analysis of the chemical compound. Such Td 5% may be obtained through the TGA analysis and TGA (Thermogravimetric analysis) analysis method is described below in “3. Analysis of Thermal Decomposition Temperature (Td 5%)” in the present specification.

The chemical compound can be obtained through a known method for synthesizing organic compounds and a deuterium substitution method. The chemical compound of Chemical Formula 1 and/or the structure of the Chemical Formulas 6 to 9 are the structures of absorbents known as so-called squarylium compounds. Various methods for preparing squarylium compounds are known in the art. Thus, for example, the chemical compound can be prepared by, for example, substituting a reactant used in a known process for preparing a squarylium-based absorbent with deuterium and applying the reactant to a process for synthesizing the squarylium-based absorbent. In addition, the chemical compound can also be prepared by, for example, synthesizing a squarylium-based absorbent according to a known synthetic method, and then, replacing at least some or all of the hydrogen in the synthesized absorbent with deuterium.

There is no special limitation on the deuterium substitution method mentioned above. For example, a method of mixing deuterium of a chemical compound to be substituted with deuterium at an appropriate temperature can be applied. The lower limit of the mixing temperature mentioned above can be, for example, about 90° C., 95° C., 100° C., 105° C., 110° C., 115° C., or 120° C. and the upper limit can be about 300° C., 280° C., 260° C., 240° C., 220° C., 200° C., 180° C., 160° C., 140° C., or 120° C. The mixing temperature may be within a range that is equal to or greater than any one of the lower limits described above; within a range that is less than or equal to any one of the upper limits described above; or within a range that is equal to or greater than any one of the lower limits described above and at the same time less than or equal to any one of the upper limits described above.

The above mixing time is not particularly limited and can be adjusted in consideration of, for example, the desired substitution rate, etc. For example, the lower limit of the mixing time can be about 3 hours, 6 hours, 9 hours, 12 hours, 15 hours, 18 hours, 21 hours, or 24 hours and the upper limit can be about 72 hours, 36 hours, or 24 hours. The mixing time can be within a range that is equal to or greater than any one of the lower limits described above; within a range that is less than or equal to any one of the upper limits described above; or within a range that is equal to or greater than any one of the lower limits described above and less than or equal to any one of the upper limits described above.

Additionally, the mixing may be carried out, for example, in the presence of additional chemical compounds capable of assisting, promoting or initiating the substitution with deuterium. For example, chemical compounds that act as a catalyst for substitution with deuterium may be any one of silver oxide (Ag₂O), silver acetate (AgOAc), silver trifluoroacetate (CFCOOAg, silver carbonate (Ag₂CO₃), palladium acetate (Pd(OAc)₂), palladium chloride (PdCl₂), bis(acetonitrile)dichloropalladium (PdCl₂(CH₃CN)₂), tris(dibenzylideneacetone) dipalladium (Pd₂(dba)₃), tetrakis(triphenylphosphine) palladium (Pd(PPh₃)₄), and bis(dibenzylideneacetone) palladium (Pd (dba) 2), but are not limited as long as they are the chemical compounds that can act as a catalyst for deuterium substitution. Also, when deuterium is substituted, the chemical compounds that act as ligands may include any one of, for example, allyldiphenylphosphine, allyldiphenylphosphine oxide, benzyldiphenylphosphine, 1-[2-[bis(tert-butyl) phosphino]phenyl]-3,5-diphenyl-1H-pyrazole, bis[2-(diadamantylphosphino)ethyl]amine, bis(5H-dibenzo[a,d]cyclohepten-5-yl)phenylphosphine, bis(5H-dibenzo[a,d]cyclohepten-5-yl)phenylphosphine, 2-[Bis(3,5-di-tert-butyl-4-methoxyphenyl)phosphino]benzaldehyde, 2,6-bis(di-tert-butylphosphinomethyl)pyridine, bis(dicyclohexylphosphinophenyl) ether, bis(diethylamino)phenylphosphine, 1,3-bis-(2,6-diisopropylphenyl)-[1,3,2]diazaphospholidine 2-oxide, bis(dimethylamino) chlorophosphine, 2-[bis(3,5-dimethylphenyl)phosphino]benzaldehyde, 2,2′-bis(diphenylphosphino)-1,1′-biphenyl, bis(4-fluorophenyl)phenylphosphine oxide, bis[4-(3,3,4,4,5,5,5-heptafluoro-2,2-bis(trifluoromethyl) pentyl)phenyl]phenylphosphine, 1,1′-bis(phenylphosphinidene) ferrocene, (2-bromophenyl)dicyclohexylphosphine, (2-bromophenyl)diphenylphosphine, tert-butyldicyclohexylphosphine, tert-butyldiisopropylphosphine, tert-butyldiphenylphosphine, di-tert-butyl(2,2-diphenyl-1-methyl-1-cyclopropyl)phosphine, 2-chloro-1,3-bis(2,6-diisopropylphenyl)-1,3,2-diazaphospholidine, 2-dicyclohexylphosphino-2′,6′-bis(N,N-dimethylamino) biphenyl, 1-(dicyclohexylphosphino)-2,2-diphenyl-1-methylcyclopropane, dicyclohexyl(2,2-diphenyl-1-methyl-1-cyclopropyl)phosphine, cyclohexyldiphenylphosphine, 2-(dicyclohexylphosphino)-1,1-diphenyl-1-propene, dicyclohexyl(1-methyl-2,2-diphenylvinyl)phosphine, di(1-adamantyl)-2-dimethylaminophenylphosphine, di-1-adamantylphosphine, di(1-adamantyl)-(2-triisopropylsiloxyphenyl)phosphine, (5H-dibenzo[a,d]cyclohepten-5-yl)diphenylphosphine, (R)-(−)-1-[(S)-2-(di(3,5-bis-trifluoromethylphenylphosphino) ferrocenyl]ethyldicyclohexylphosphine, (R)-(−)-1-[(S)-2-(di(3,5-bis-trifluoromethylphenyl)phosphino) ferrocenyl]ethyldi(3,5-dimethylphenyl)phosphine, P,P-dichloroferrocenylphosphine, (R)-(−)—N,N-dimethyl-1-[(S)-2-(diphenylphosphino) ferrocenyl]ethylamine, and 1,2,3,4,5-pentaphenyl-1′-(di-tert-butylphosphino) ferrocene. However, it is not limited to any chemical compound that can act as a ligand upon deuterium substitution.

The present specification also discloses a chemical composition comprising a composition. The term “composition” may mean a mixture comprising the chemical compound and other components or a mixture comprising two or more chemical compounds. A composition comprising such a chemical compound basically comprises the chemical compound disclosed in the present specification and may additionally comprise other necessary components.

For example, the composition may additionally include a resin component that acts as a binder. In this case, there is no particular limitation on the type of the resin component applied, and a known resin component used for forming a resin film, for example, a near-infrared resin film, may be applied. In the present specification, the chemical compound component may exhibit appropriate compatibility or solubility with respect to various known resin components.

Examples of the resin component may include, but are not limited to, cyclic olefin (COP) resin, polyarylate resin, polyester resin, polysulfone resin, polyether sulfone resin, polyparaphenylene resin, polyarylene ether phosphine oxide resin, polyimide resin, polyetherimide resin, polyamideimide resin, acrylic resin, polycarbonate resin, polyethylene naphthalate resin, or silicone resin or at least one of various organic resins or organic-inorganic hybrid series resins. Although not particularly limited, the chemical compound of the present specification may be mixed with a cyclic olefin resin among the resin components that serve as binders known in the art to form a resin film exhibiting excellent performance. Accordingly, in one example, the resin component may be a cyclic olefin resin.

When the resin component is applied, there is no special limitation for the ratio. For example, the resin component may be present so that the weight ratio of the chemical compound to 100 parts by weight of the resin component may be within a range of about 0.001 parts by weight to 10 parts by weight. The lower limit of the weight ratio of the chemical compound to 100 parts by weight of the resin component may be, in other examples, about 0.001 parts by weight, 0.005 parts by weight, 0.01 parts by weight, 0.05 parts by weight, 0.1 parts by weight, 0.5 parts by weight, 1 part by weight, 1.1 parts by weight, 1.2 parts by weight, 1.3 parts by weight, or 1.4 parts by weight and the upper limit may be about 10 parts by weight, 9 parts by weight, 8 parts by weight, 7 parts by weight, 6 parts by weight, 5 parts by weight, 4 parts by weight, 3 parts by weight, 2 parts by weight, or 1.5 parts by weight. The above ratio may be within a range that is equal to or greater than any one of the lower limits described above; within a range that is less than or equal to any one of the upper limits described above; or within a range that is equal to or greater than any one of the lower limits described above and less than or equal to any one of the upper limits described above.

For example, the composition may additionally include a solvent in which the compound and/or the resin component is dispersed. In this case, there is no particular limitation on the type of solvent applied and a known solvent used for forming a resin film, for example, a near-infrared resin film, may be applied. The chemical compound component in the present specification may exhibit appropriate compatibility or solubility with respect to various known solvents.

Examples of solvents may include methylene chloride, cyclohexanone, toluene, methyl ethyl ketone, methyl isobutyl ketone, propylene glycol methyl ether acetate, diethylene glycol monoethyl ether 3-methoxy butanol, ethylene glycol monobutyl ether acetate, 4-hydroxy-4-methyl-2-pentanone, gamma butyrolactone, cyclohexanone, pyridone, chloroform, 1,4-dioxane, cyclohexanone, ortho-dichlorobenzene, aliphatic alcohols having 2 or more carbon atoms of chlorobenzene (e.g., isobutyl alcohol, isopropyl alcohol, ethanol, isopropanol, butanol, etc.), butyl acetate, tetrahydrofuran, or xylene. However, the examples are not limited to.

There is no special limitation on the ratio when a solvent is applied. The ratio can be adjusted within a range where appropriate dispersion of the chemical compound and/or resin component is possible.

The composition may contain other optional components in addition to the components described above. The optional components may include, for example, adhesives. These include, but are not limited to, binders, leveling agents, antistatic agents, heat stabilizers, light stabilizers, antioxidants, dispersants, flame retardants, lubricants, or plasticizers.

The present invention also relates to uses of the chemical compound or the composition. For example, the present invention may relate to a resin film where the chemical compound or the composition is applied. Such a resin film may comprise at least a resin component and the chemical compound. The specific type of resin component and the ratio of the resin component and the chemical compound for these cases are as described in the section for the chemical compound composition.

The resin film may be a film capable of absorbing light within a predetermined range of wavelengths. In one example, the resin film may be an infrared resin film or a near-infrared resin film. Such a resin film, for example, may exhibit absorption characteristics in at least a portion of a wavelength range within a range of about 650 nm to 900 nm.

For example, the resin film can exhibit an absorption maximum wavelength within a range of 650 nm to 900 nm. The lower limit of the absorption maximum wavelength can be, in other examples, about 650 nm, 660 nm, 670 nm, 680 nm, 690 nm, 700 nm, 710 nm, 720 nm, 730 nm, 740 nm, 750 nm, 760 nm, or 765 nm. In addition, the upper limit of the absorption maximum wavelength can be about 900 nm, 800 nm, 790 nm, 780 nm, 770 nm, 760 nm, 750 nm, 740 nm, 730 nm, 720 nm, or 710 nm. The absorption maximum wavelength may be within a range that is equal to or greater than any one of the lower limits described above; within a range that is less than or equal to any one of the upper limits described above; or within a range that is equal to or greater than any one of the lower limits described above and at the same time less than or equal to any one of the upper limits described above. Due to these characteristics, the resin film can be applied to various devices such as optical filters and infrared sensors, and can have excellent optical characteristics and physical properties such as excellent heat resistance.

For example, the transmittance at the absorption peak of the resin film may be below a certain level. The upper limit may be about 50%, 49%, 48%, 47%, 46% 45%, 45%, 44%, 43%, 42%, 41%, or 40% and the lower limit may be about 0.1%, 1%, 10%, 15%, 20%, 25%, 30%, 35%, or 39%. The transmittance at the absorption peak may be within a range that is equal to or greater than any one of the lower limits described above; within a range that is less than or equal to any one of the upper limits described above; or within a range that is equal to or greater than any one of the lower limits described above and less than or equal to any one of the upper limits described above.

For example, the absolute value of ΔA in Equation 1 of the resin film may be less than or equal to a predetermined value:

$$△A = 100 \times ( {A}_{f} - {A}_{i} ) / {A}_{i} , \qquad [ Equation 1 ]$$

In Equation 1, A_{f} is the transmittance at the maximum absorption wavelength of the resin film maintained at 85° C. and a relative humidity of 85% for 120 hours. Aᵢ is the transmittance at the maximum absorption wavelength of the resin film before being maintained at 85° C. and a relative humidity of 85% for 120 hours. The maximum absorption wavelength exists within a wavelength range of 650 nm to 900 nm.

The upper limit of the absolute value of ΔA in Equation 1 may be about 50%, 40%, 30%, 20%, 10%, 9%, 8%, 7%, 6%, 5%, 4%, 3%, 2%, 1.5%, 1.4%, 1.3%, 1.2%, 1.1%, 1%, 0.9%, 0.8%, 0.7%, 0.6%, 0.5%, 0.4%, 0.3%, 0.2%, 0.1%, or 0%. The lower limit is not particularly limited, but may be about 0% or 0.1%. The absolute value of ΔA may be within a range that is less than or equal to any one of the upper limits described above or within a range that is equal to or greater than any one of the lower limits described above and at the same time less than or equal to any one of the upper limits described above.

In Equation 1, the upper limit of A_{f} can be about 20%, 19%, 18%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1% and the lower limit can be about 0.1%, 0.2%, 0.3%, 0.4%, 0.5%, 0.6%, 0.7%, 0.8%, 0.9%, or 1%. A_{f} may be within a range that is less than or equal to any one of the upper limits described above or within a range that is equal to or greater than any one of the lower limits described above and at the same time less than or equal to any one of the upper limits described above.

The resin film may have an absolute value of Δλ in Equation 2 below that is equal to or less than a predetermined value:

$$△λ = 100 \times ( {λ}_{f} - {λ}_{i} ) / {λ}_{i} , \qquad [ Equation 2 ]$$

In Equation 2, A_{f} is the maximum absorption wavelength of the resin film maintained at 85° C. and a relative humidity of 85% for 120 hours. Aᵢ is the maximum absorption wavelength of the resin film before being maintained at 85° C. and a relative humidity of 85% for 120 hours. The maximum absorption wavelength exists within a wavelength range of 650 nm to 900 nm.

The upper limit of the absolute value of Δλ in Equation 2 may be about 10%, 9%, 8%, 7%, 6%, 5%, 4%, 3%, 2%, 1%, or 0.5% and its lower limit may be 0%. The absolute value of Δλ may be within a range that is less than or equal to any one of the above-described upper limits or within a range that is equal to or greater than any one of the above-described lower limits and at the same time less than or equal to any one of the above-described upper limits.

In Equation 2, λ_{f} and λᵢ may each be in a range of 650 nm to 900 nm. The lower limit of each of A_{f} and 2; may be about 650 nm, 660 nm, 670 nm, 680 nm, 690 nm, 700 nm, 710 nm, 720 nm, 730 nm, 740 nm, 750 nm, 760 nm, or 765 nm. In addition, the upper limit of each of A_{f} and λᵢ may be about 900 nm, 800 nm, 790 nm, 780 nm, 770 nm, 760 nm, 750 nm, 740 nm, 730 nm, 720 nm, or 710 nm. Each of the above A_{f} and Aᵢ may be within a range that is equal to or greater than any one of the lower limits described above; within a range that is less than or equal to any one of the upper limits described above; or within a range that is equal to or greater than any one of the lower limits described above and at the same time less than or equal to any one of the upper limits described above. Through the above absorption characteristics, the resin film can be applied to various devices such as optical filters and infrared sensors to efficiently achieve the desired characteristics.

In the present specification, the resin film can be formed by a known method as long as the chemical compound or composition of the present specification is applied. For example, the resin film can be formed by coating the chemical compound composition in an appropriate manner and performing a curing or drying process, if necessary.

There is no particular limitation on the thickness of the resin film. The thickness can be adjusted in consideration of the desired characteristics. In one example, the resin film can have a thickness within a range of approximately 0.5 to 20 μm.

The present specification also relates to an optical filter. The optical filter may include a substrate layer and the resin film formed on one or both sides of the substrate layer.

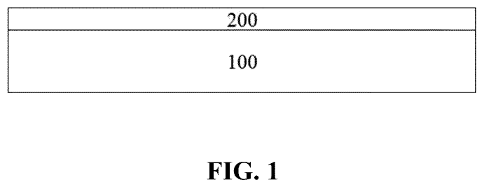

FIG. 1 is a drawing showing an example of the optical filter where the resin film 200 is formed on one surface of the substrate 100. The optical filter can exhibit excellent performance by including the above-described resin film, for example, the optical filter can implement a visible light transmission band with high transmittance while efficiently and accurately blocking unnecessary infrared light.

There is no particular limitation on the type of transparent substrate applied to the optical filter. Any known transparent substrate for optical filters can be used.

In one example, the substrate layer may be a so-called near-infrared absorbing substrate. A near-infrared absorbing substrate is a substrate that exhibits absorption characteristics in at least a portion of a near-infrared region. So-called a blue glass, which exhibits the characteristics by including copper, is a representative example of the near-infrared absorbing substrate. Such a near-infrared absorbing substrate is useful in configuring an optical filter that blocks light in the near-infrared region. It is disadvantageous, however, in securing high transmittance in the visible light region due to the absorption characteristics and is also disadvantageous in terms of durability. In the present specification, by selecting a near-infrared absorbing substrate and combining it with a specific resin film, it is possible to provide an optical filter that efficiently blocks desired light, exhibits high transmittance characteristics in the visible light region, and has excellent durability.

As the infrared absorbing substrate, a substrate showing an average transmittance of a certain level or higher within a range of 425 nm to 560 nm can be used. The lower limit of the average transmittance may be about 75%, 77%, 79%, 81%, 83%, 85%, 87%, or 89% and the upper limit may be about 98%, 96%, 94%, 92%, or 90%. The average transmittance may be within a range that is equal to or greater than any one of the lower limits described above or within a range that is equal to or greater than any one of the lower limits described above and at the same time less than or equal to any one of the upper limits described above.

As the infrared absorbing substrate, a substrate showing a maximum transmittance of a certain level or higher within a range of 425 nm to 560 nm can be used. The lower limit of the maximum transmittance may be about 80%, 82%, 84%, 86%, 88%, or 90% and the upper limit may be about 100%, 98%, 96%, 94%, 92%, or 90%. The maximum transmittance may be within a range that is equal to or greater than any one of the lower limits described above or within a range that is equal to or greater than any one of the lower limits described above and at the same time less than or equal to any one of the upper limits described above.

As the infrared absorbing substrate, a substrate that exhibits an average transmittance of a certain level or higher within a range of 350 nm to 390 nm can be used. The lower limit of the average transmittance may be about 75%, 77%, 79%, 81%, or 83% and the upper limit may be about 98%, 96%, 94%, 92%, 90%, 88%, 86%, or 84%. The average transmittance may be within a range that is equal to or greater than any one of the lower limits described above or within a range that is equal to or greater than any one of the lower limits described above and at the same time less than or equal to any one of the upper limits described above.

As the infrared absorbing substrate, a substrate showing a maximum transmittance of a certain level or higher within a range of 350 nm to 390 nm can be used. The lower limit of the maximum transmittance may be about 80%, 82%, 84%, 86%, or 87% and the upper limit may be about 100%, 98%, 96%, 94%, 92%, 90%, or 88%. The maximum transmittance may be within a range that is equal to or greater than any one of the lower limits described above or within a range that is equal to or greater than any one of the lower limits described above and at the same time less than or equal to any one of the upper limits described above.

The infrared absorbing substrate may have a transmittance at a wavelength of 700 nm within a certain range. The lower limit may be about 10%, 12%, 14%, 16%, 18%, 20%, 22%, 24%, 26%, or 28% and the upper limit may be about 45%, 43%, 41%, 39%, 37%, 35%, 33%, 31%, or 29%. The transmittance may be within a range that is equal to or greater than any one of the lower limits described above; within a range that is less than or equal to any one of the upper limits described above; or within a range that is equal to or greater than any one of the lower limits described above and less than or equal to any one of the upper limits described above.

The infrared absorbing substrate may have an average transmittance within a range of 700 nm to 800 nm. The lower limit may be about 5%, 7%, 9%, 11%, 13%, 15%, 15.5%, 16%, or 16.5% and the upper limit may be about 30%, 28%, 26%, 24%, 22%, 20%, 18%, or 17%. The average transmittance may be within a range that is equal to or greater than any one of the lower limits described above; within a range that is less than or equal to any one of the upper limits described above; or within a range that is equal to or greater than any one of the lower limits described above and less than or equal to any one of the upper limits described above.

The infrared absorbing substrate may have a maximum transmittance within a range of 700 nm to 800 nm. The lower limit may be about 10%, 12%, 14%, 16%, 18%, 20%, 22%, 24%, 26%, or 28% and the upper limit may be about 43%, 41%, 39%, 37%, 35%, 33%, 31%, or 29%. The maximum transmittance may be within a range that is equal to or greater than any one of the lower limits described above; within a range that is less than or equal to any one of the upper limits described above; or within a range that is equal to or greater than any one of the lower limits described above and less than or equal to any one of the upper limits described above.

The infrared absorbing substrate may have an average transmittance within a range of 800 nm to 1,000 nm. The lower limit may be about 3%, 5%, 7%, 9%, or 11% and the upper limit may be about 20%, 18%, 16%, 14%, or 12%. The average transmittance may be within a range that is equal to or greater than any one of the lower limits described above; within a range that is less than or equal to any one of the upper limits described above; or within a range that is equal to or greater than any one of the lower limits described above and less than or equal to any one of the upper limits described above.

The infrared absorbing substrate may have a maximum transmittance within a range of 800 nm to 1,000 nm. The lower limit may be about 5%, 7%, 9%, 11%, 13%, or 15% and the upper limit may be about 30%, 28%, 26%, 24%, 22%, 20%, 18%, or 16%. The maximum transmittance may be within a range that is equal to or greater than any one of the lower limits described above; within a range that is less than or equal to any one of the upper limits described above; or within a range that is equal to or greater than any one of the lower limits described above and less than or equal to any one of the upper limits described above.

The infrared absorbing substrate may have an average transmittance within a range of 1,000 nm to 1,200 nm. The lower limit may be about 10%, 12%, 14%, 16%, 18%, 20%, 22%, 24%, or 25% and the upper limit may be about 50%, 48%, 46%, 44%, 42%, 40%, 38%, 36%, 34%, 32%, 30%, 28%, or 26%. The average transmittance may be within a range that is equal to or greater than any one of the lower limits described above; within a range that is less than or equal to any one of the upper limits described above; or within a range that is equal to or greater than any one of the lower limits described above and less than or equal to any one of the upper limits described above.

The infrared absorbing substrate may have a maximum transmittance within a range of 1,000 nm to 1,200 nm. The lower limit may be about 10%, 12%, 14%, 16%, 18%, 20%, 22%, 24%, 26%, 28%, 30%, 32%, 34%, or 36% and the upper limit may be about 70%, 68%, 66%, 64%, 62%, 60%, 58%, 56%, 54%, 52%, 50%, 48%, 46%, 44%, 42%, 40%, 38%, or 36%. The above maximum transmittance may be within a range that is equal to or greater than any one of the lower limits described above; within a range that is less than or equal to any one of the upper limits described above; or within a range that is equal to or greater than any one of the lower limits described above and at the same time less than or equal to any one of the upper limits described above.

The infrared absorbing substrate can be combined with the resin film of the present specification to form a desired optical filter. As such a substrate, a substrate known as so-called infrared absorbing glass can be used. Such glass is an absorption glass manufactured by adding CuO or the like to fluorophosphate glass or phosphate glass. Therefore, in one example, in the present specification, as for the infrared absorbing substrate, a CuO-containing fluorophosphate glass substrate or a CuO-containing phosphate glass substrate can be used. The phosphate glass mentioned above also includes K-phosphate glass where a portion of the glass skeleton is composed of SiO₂. Such absorption glass is known. For example, glasses disclosed in Korean Patent No. 10-2056613, etc., or other commercially available absorption glasses (for example, commercial products from HOYA, SCHOTT, PTOT, etc.) can be used.

These infrared absorbing substrates contain copper. In the present specification, a substrate having a copper content of 1 wt % to 7 wt % can be used. The copper content may be about 1.5 wt % or more, 2 wt % or more, 2.5 wt % or more, 2.6 wt % or more, 2.7 wt % or more, or 2.8 wt % or more or in other examples, it may be about 6.5 wt % or less, 6 wt % or less, 5.5 wt % or less, 5 wt % or less, 4.5 wt % or less, 4 wt % or less, 3.5 wt % or less, 3 wt % or less, or 2.9 wt % or less. A substrate having such a copper content tends to exhibit the optical properties described above and can be combined with the resin film to form an optical filter having desired properties.

The above copper content can be confirmed using X-ray fluorescence analysis equipment (WD XRF, Wavelength Dispersive X-Ray Fluorescence Spectrometry). When X-rays are irradiated to a specimen (substrate) using the above equipment, characteristic secondary X-rays are generated from individual elements of the specimen and the equipment detects the secondary X-rays according to the wavelength of each element. The intensity of the secondary X-rays is proportional to the element content, and therefore, quantitative analysis can be performed through the intensity of the secondary X-rays measured according to the wavelength of each element.

The thickness of the above infrared absorbing substrate can be controlled within a range of, for example, about 0.03 mm to 5 mm. However, it is not limited to the range.

The optical filter of the present specification may also include other known components additionally required for the substrate and resin film. For example, the optical filter may additionally include a dielectric film. The dielectric film may additionally include a so-called dielectric film, for example, on one side or both sides of the substrate.

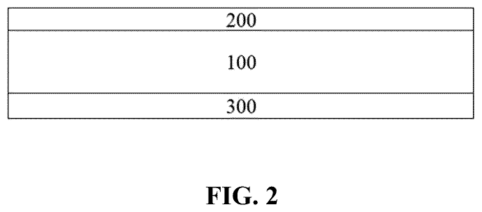

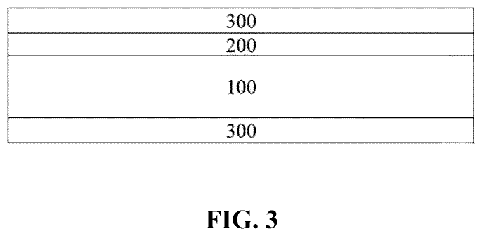

FIGS. 2 and 3 are examples of optical filters with an added dielectric film 300. They show a case where the dielectric film 300 is formed on one or both sides of a laminated structure including a substrate 100 and a resin film 200.

This dielectric film is a film formed by repeatedly laminating a low refractive index dielectric material and a high refractive index dielectric material and is used to form a so-called IR reflection layer and AR (anti-reflection) layer. In the present specification, a dielectric film for forming such a known IR reflection layer or AR layer can be applied.

Accordingly, the dielectric film may have a multilayer structure including at least two types of sublayers, each having a different refractive index, and may include a multilayer structure where the two types of sublayers are repeatedly stacked.

The type of material forming the dielectric film, i.e., the material forming each sublayer, is not particularly limited, and known materials can be applied. Typically, for the preparation of a low-refractive sublayer, fluorides such as SiO₂ or Na₅Al₃F₁₄, Na₃AlF₆, or MgF₂ are applied. For the preparation of a high-refractive sublayer, amorphous silicon, TiO₂, Ta₂O₅, Nb₂O₅, ZnS, or ZnSe can be applied, but the materials applied in the present specification are not limited to.

The method of forming the dielectric film as described above is not particularly limited, and for example, it can be formed by applying a known deposition method. In the industry, a method of controlling the reflection and transmission characteristics of the dielectric film by considering the deposition thickness or number of layers of the sublayer is known. In the present specification, the dielectric film can be formed according to such a known method.

In addition, the optical filter may additionally include a resin film (“UV resin film”) that exhibits absorption characteristics for ultraviolet rays as a resin film distinct from the resin film. However, such a resin film is not an essential component, and for example, an ultraviolet compound described below may be introduced into a single resin film together with a compound disclosed in the present specification. In one example, the UV resin film can be designed to exhibit an absorption peak in a wavelength range of about 300 nm to 390 nm.

The UV resin film may contain only an ultraviolet compound or may contain two or more types of ultraviolet compounds, if necessary. For example, as an ultraviolet compound, a known compound showing an absorption maximum in a wavelength range of about 300 nm to 390 nm can be applied.

The materials and composition methods constituting the UV resin film are not particularly limited and known materials and composition methods can be applied. Typically, an ultraviolet resin film is formed using a material in which an ultraviolet compound capable of exhibiting a desired absorption peak is mixed with a transparent resin. At this time, a resin component applied to the compound composition may be applied to the transparent resin. In addition to the layers described above, optical filters may include various layers as needed, as long as the desired effect is not impaired.

The present specification also relates to an imaging capturing device including the optical filter. The configuration of the imaging capturing device or the application of the optical filter are not particularly limited and known configurations and application methods can be applied. Furthermore, the use of the optical filter of the present specification is not limited to the imaging capturing device and may be applied to various other applications requiring near-infrared cutoff, such as display devices such as PDPs.

The present specification also relates to an infrared sensor including the resin film. The configuration of the infrared sensor is not particularly limited as long as the resin film is included. For example, the resin film can be incorporated into a known motion sensor, proximity sensor, or gesture sensor. Furthermore, the use of the chemical compound composition or resin film of the present specification is not limited to the optical filter, infrared sensor, and/or imaging device and may be applied to various other applications requiring infrared cutoff, such as automotive components such as LiDAR.

The chemical compounds disclosed in the present specification are specifically described through the following Embodiments and Comparative Examples. The scope of the chemical compounds, however, is not limited by Embodiments.

### 1. Method for Measuring Absorption Maximum Wavelength

The maximum absorption wavelength of the chemical compound was evaluated by a conventional method. Specifically, the sample (chemical compound) was dissolved in chloroform at a concentration of about 10⁻⁵ M, and then, the maximum absorption wavelength was evaluated using a measuring device (Agilent, Varian Cary 4000).

### 2. Evaluation of Transmittance Spectrum

The transmittance spectrum was measured using a spectrophotometer (Perkinelmer, Lambda750 spectrophotometer) on specimens obtained by cutting the measurement target (e.g., resin film) to be 10 mm wide and 10 mm long, respectively. The transmittance spectrum was measured for each wavelength according to the manual of the above equipment. The specimen was positioned on a straight line between the measurement beam and detector of the spectrophotometer and the transmittance spectrum was confirmed with the measurement beam having an incident angle of 0 degrees. The incident angle of 0 degrees is a direction substantially parallel to the normal direction of the surface of the specimen.

The average transmittance within a given wavelength range in the transmittance spectrum is the result of calculating the arithmetic mean of the transmittances measured while increasing the wavelength by 1 nm from the shortest wavelength in the wavelength range and then measuring the transmittance at each wavelength. The maximum transmittance is the highest transmittance among the transmittances measured while increasing the wavelength by 1 nm and the minimum transmittance is the lowest transmittance among the transmittances measured while increasing the wavelength by 1 nm. For example, the average transmittance within the wavelength range of 350 nm to 360 nm is the arithmetic mean of the transmittances measured at wavelengths of 350 nm, 351 nm, 352 nm, 353 nm, 354 nm, 355 nm, 356 nm, 357 nm, 358 nm, 359 nm, and 360 nm. The maximum transmittance within the wavelength range of 350 nm to 360 nm is the highest transmittance among the transmittances measured at wavelengths of 350 nm, 351 nm, 352 nm, 353 nm, 354 nm, 355 nm, 356 nm, 357 nm, 358 nm, 359 nm, and 360 nm and the minimum transmittance within the wavelength range of 350 nm to 360 nm is the lowest transmittance among the transmittances measured at wavelengths of 350 nm, 351 nm, 352 nm, 353 nm, 354 nm, 355 nm, 356 nm, 357 nm, 358 nm, 359 nm, and 360 nm.

### 3. Analysis of Thermal Decomposition Temperature (Td 5%)

Thermogravimetric analysis (TGA) of the chemical compound was performed using a TGA N-1000 device from Scinco. The analysis was performed using approximately 3 mg of the sample (chemical compound) under the conditions of a temperature range of 25° C. to 800° C., a heating rate of 10° C./min, and a nitrogen (N₂) atmosphere of 60 cm³/min. Td decomposition temperature was used as the value at 95% of the weight loss (Td 5%).

### 4. Mass Analysis (Maldi-Tof)

Mass analysis of the chemical compound was performed using a MALDI-TOF Voyager DE-STR instrument (Applied Biosystems, USA) measured in positive mode from reflector mode and analyzed using dithranol matrix.

### 5. Deuterium Substitution Rate

The deuterium substitution rate of the chemical compound was measured through hydrogen nuclear magnetic resonance (¹H-NMR) analysis. ¹H-NMR analysis was performed on the chemical compound substituted with deuterium (sample chemical compound) and the chemical compound of the same structure before deuterium substitution (reference chemical compound). ¹H-NMR analysis of the reference chemical compound was performed to confirm the position and area of the hydrogen peak of the chemical compound and ¹H-NMR analysis of the sample chemical compound was performed to confirm the position and area of the non-deuterium hydrogen peak and the deuterium peak of the chemical compound. The deuterium substitution rate was confirmed by comparing the peak positions and areas.

### The Substitution Rate was Calculated by Using Equation A:

$$Substitution rate = 100 \times D / H . \qquad [ Equation A ]$$

In Equation A, D represents the integral of the deuterium peak in the ¹H-NMR analysis of the sample chemical compound. H represents the integral of the hydrogen peak in the ¹H-NMR analysis of the reference chemical compound.

Meanwhile, the ¹H NMR analysis was performed using JEOL's JNM-ECX400 by dissolving the sample or reference chemical compound in CDCl₃ containing tetramethylsilane (TMS). Chemical shifts are expressed in ppm.

### Embodiment 1

### Chemical Compound a of Chemical Formula a was Synthesized by the Following Method:

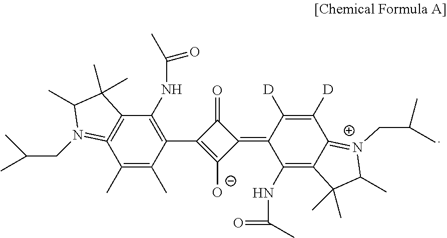

In Chemical Formula A, D is hydrogen or deuterium. However, at least one of the Ds is deuterium.

Chemical Compound A was synthesized using Chemical Compound E of Chemical Formula E of Comparative Example 1. 0.01 mol of Chemical Compound E of Comparative Example 1, 0.54 g of silver carbonate (Ag₂CO₃), and 1.34 g of cyclohexyldiphenylphosphine were placed in a three-necked flask equipped with a reflux device. Subsequently, 0.1 mL of toluene and excessive amount of deuterium oxide were added. Then, the mixture was stirred at about 120° C. for 24 hours. Thereafter, 10 mL of dichloromethane and 5 mL of water were added to the flask, and then, the mixture was stirred and mixed for an additional 30 minutes. The dichloromethane layer was separated from the mixture using a separatory funnel and then, methanol was added to obtain the target chemical compound (Chemical Compound A) through recrystallization (Maldi-tof m/z 630.01 [M+H]⁺). The deuterium substitution rate of Chemical Compound A of Embodiment 1 was approximately 79.1%.

### Embodiment 2

### Chemical Compound B of Chemical Formula B was Synthesized by the Following Method:

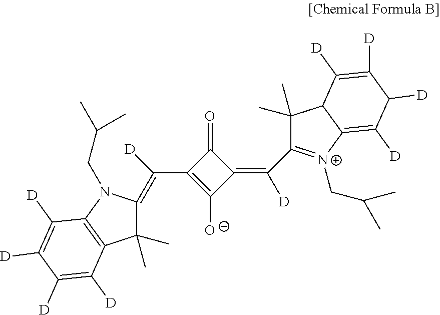 [SMILES: [2H]C1=C([2H])C2C(=C([2H])C1[2H])[N+](CC(C)C)=C(/C([2H])=C1\C(=O)C(C(/[2H])=C3/N(CC(C)C)c4c([2H])c([2H])c([2H])c([2H])c4C3(C)C)=C1[O-])C2(C)C]

In Chemical Formula B, D is hydrogen or deuterium, but at least one of the Ds is deuterium. Chemical Compound B was synthesized in the same manner as Embodiment 1 except that Chemical Compound F of Chemical Formula F of Comparative Example 2 was used instead of Chemical Compound E of Comparative Example 1 used for Embodiment 1 (Maldi-tof m/z 518.19 [M+H]⁺). The deuterium substitution rate of the Chemical Compound B of Embodiment 2 was approximately 74.2%.

### Embodiment 3

### Chemical Compound C of Chemical Formula C was Synthesized by the Following Method:

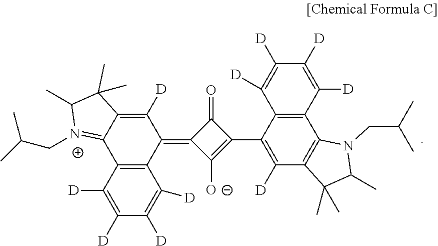 [SMILES: [2H]c1c([2H])c([2H])c2c3c(c([2H])c(C4=C([O-])/C(=c5/c([2H])c6c(c7c([2H])c([2H])c([2H])c([2H])c57)=[N+](CC(C)C)C(C)C6(C)C)C4=O)c2c1[2H])C(C)(C)C(C)N3CC(C)C]

In Chemical Formula C, D is hydrogen or deuterium, but at least one of the Ds is deuterium. Chemical compound C was synthesized in the same manner as in Embodiment 1 except that Chemical Compound G of Comparative Example 3 was used instead of Chemical Compound E of Comparative Example 1 for Embodiment 1 (Maldi-tof m/z 622.03 [M+H]⁺). The deuterium substitution rate of Chemical Compound C of Embodiment 3 was approximately 91.3%.

### Embodiment 4

### Chemical Compound D of Chemical Formula D was Synthesized by the Following Method:

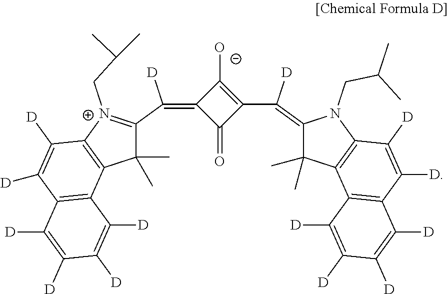

In Chemical Formula D, D is hydrogen or deuterium, but at least one of the Ds is deuterium. Chemical Compound D was synthesized in the same manner as in Embodiment 1 except that Chemical Compound H of Comparative Example 4 was used instead of Chemical Compound E of Comparative Example 1 (Maldi-tof m/z 621.57 [M+H]⁺). The deuterium substitution rate of Chemical Compound D of Embodiment 4 was approximately 90.5%.

### Comparative Example 1

### Chemical Compound E of Chemical Formula E was Synthesized by the Following Method:

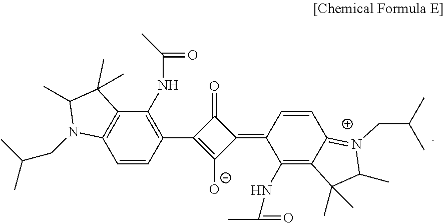 [SMILES: CC(=O)Nc1c(C2=C([O-])/C(=c3/ccc4c(c3NC(C)=O)C(C)(C)C(C)[N+]=4CC(C)C)C2=O)ccc2c1C(C)(C)C(C)N2CC(C)C]

1 g of squaric acid, 4 g of N-(1-isobutyl-2,3,3-trimethyl-1-2,3,6,7-tetrahydro-1H-indol-4-yl) acetamide, and 4 g of triethyl orthoformic acid was placed in a beaker; 20 mL of n-butanol was added as a solvent; and the mixture was stirred at about 90° C. for about 3 hours. After that, the reaction mixture was cooled using an ice bath for about 60 minutes, and then, 40 mL of ethanol was added to obtain the target chemical compound (Chemical compound E) (Maldi-tof m/z 626.34 [M+H]⁺). The deuterium substitution rate of the Chemical Compound E of Comparative Example 1 was 0%.

### Comparative Example 2

### Chemical Compound F of Chemical Formula F was Synthesized by the Following Method:

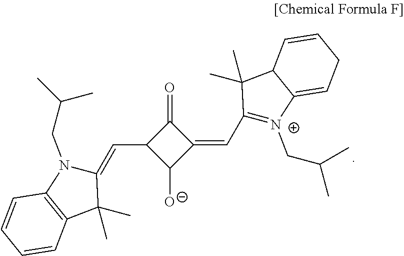 [SMILES: CC(C)CN1/C(=C/C2C(=O)/C(=C\C3=[N+](CC(C)C)C4=CCC=CC4C3(C)C)C2[O-])C(C)(C)c2ccccc21]

1 g of squaric acid, 5.5 g of 1-isobutyl-2,3,3-trimethyl-3H-indol-1-ium bromide, and 4 g of triethyl orthoformic acid was placed in a beaker; 20 mL of n-butanol was added as a solvent; and the mixture was stirred at about 90° C. for about 3 hours. The reaction mixture was cooled for 60 minutes using an ice bath and then, 40 mL of ethanol was added to obtain the target chemical compound (Chemical Compound F) (Maldi-tof m/z 508.32 [M+H]⁺). The deuterium substitution rate of the obtained chemical compound was 0%.

### Comparative Example 3

### Chemical Compound G of Chemical Formula G was Synthesized by the Following Method:

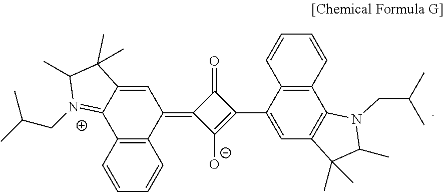 [SMILES: CC(C)CN1c2c(cc(C3=C([O-])/C(=c4/cc5c(c6ccccc46)=[N+](CC(C)C)C(C)C5(C)C)C3=O)c3ccccc23)C(C)(C)C1C]

1 g of squaric acid, 4.95 g of 1-isobutyl-2,3,3-trimethyl-2,3-dihydro-1H-benzo[g]indole, and 4 g of triethyl orthoformic acid was placed in a beaker; 20 mL of n-butanol was added as a solvent; and the mixture was stirred at about 90° C. for about 3 hours. The reaction mixture was cooled for 60 minutes using an ice bath and 40 mL of ethanol was added to obtain the target chemical compound (Chemical Compound G) (Maldi-tof m/z 612.4 [M+H]⁺). The deuterium substitution rate of the above chemical compound was approximately 0%.

### Comparative Example 4

### Chemical Compound H of Chemical Formula H was Synthesized by the Following Method:

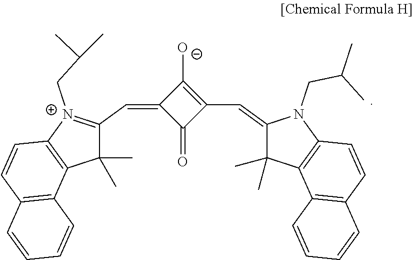 [SMILES: CC(C)CN1/C(=C/C2=C([O-])C(=C/C3=[N+](CC(C)C)c4ccc5ccccc5c4C3(C)C)/C2=O)C(C)(C)c2c1ccc1ccccc21]

1 g of squaric acid, 6.4 g of 3-isobutyl-1,1,2-trimethyl-1H-benzo[e]indol-3-ium bromide, and 4 g of triethyl orthoformic acid was placed in a beaker; 20 mL of n-butanol was added as a solvent; and the mixture was stirred at about 90° C. for about 3 hours. Then, the reaction mixture was cooled in an ice bath for 60 minutes and 40 mL of ethanol was added to obtain the target chemical compound (Chemical Compound H) (Maldi-tof m/z 608.4 [M+H]⁺). The deuterium substitution rate of the above chemical compound was 0%.

Table 1 summarizes the absorption capacity of the chemical compounds of each Embodiment and Comparative Example. In Table 1, T % (λₘₐₓ) is the transmittance at each absorption maximum wavelength confirmed in “1. Method for Measuring Absorption Maximum Wavelength.” Td 5% in Table 1 is the temperature (Td 5%) at where 95% of the weight loss of the chemical compound occurred in TGA (thermogravimetric analysis) analysis of the above “3. Analysis of Thermal Decomposition Temperature (Td 5%).”

TABLE 1

| | Absorption Peak | T % | Td 5% |
|---|---|---|---|
| | Wavelength (nm) | (λₘₐₓ) | (° C.) |
| Embodiment 1 | 709 | 0.1 | 245 |
| Embodiment 2 | 736 | 0.2 | 297 |
| Embodiment 3 | 764 | 17.7 | 278 |
| Embodiment 4 | 769 | 0.6 | 286 |
| Comparative Example 1 | 709 | 0.1 | 239 |
| Comparative Example 2 | 736 | 0.2 | 292 |
| Comparative Example 3 | 764 | 17.6 | 271 |
| Comparative Example 4 | 769 | 0.6 | 275 |

Through Table 1, it can be confirmed that each chemical compound of Embodiments 1 to 4 exhibits excellent heat resistance while exhibiting light absorption characteristics equivalent to those of Comparative Examples 1 to 4.

### Test Example 1

A coating solution was prepared by mixing a cyclic olefin resin (TOPAS, 5013F-04), a chemical compound, and a solvent (cyclohexanone). The chemical compound synthesized in the Embodiments or Comparative Examples was used as the chemical compound. The mixing ratio of the cyclic olefin resin, the chemical compound, and the solvent was approximately 69.3:0.99:29.7 of the weight ratio of the cyclic olefin resin, the chemical compound, and the solvent. The coating solution was coated on a transparent substrate (a glass substrate manufactured by SCHOTT) and maintained at 140° C. for approximately 2 hours to form a resin film with a thickness of approximately 6 μm.

Table 2 below summarizes the transmittance before and after reliability test in the visible and infrared regions of the resin film manufactured using the chemical compound of Embodiment as the chemical compound of the coating solution. Table 3 below summarizes the transmittance before and after reliability test in the visible and infrared regions of the resin film manufactured using the chemical compound of Comparative Example as the chemical compound of the coating solution.

The reliability test is an evaluation where the resin film was maintained at 85° C. and 85% relative humidity for 120 hours. In Tables 2 and 3 below, B represents the result before performing the reliability test and A represents the result after performing the reliability test. In addition, in Tables 2 and 3 below, Amax represents the transmittance at each absorption maximum wavelength confirmed in “1. Method for Measuring Absorption Maximum Wavelength.”

In Tables 2 and 3 below, Δ is the change rate (%) of each characteristic before and after the reliability test and is the result calculated by 100×(AB)/B, where A is the value indicated as A in Tables 2 and 3 below and B is the value indicated as B in Tables 2 and 3 below. In Tables 2 and 3, T is the transmittance at the corresponding wavelength, Tₘᵢₙ is the minimum transmittance within the corresponding wavelength range, and Tₐᵥₑ is the average transmittance within the corresponding wavelength range.

TABLE 2

| Wavelength | | | | | | | | | | | | | |
|---|---|---|---|---|---|---|---|---|---|---|---|---|---|
| (nm) or | | | | | | | | | | | | | |
| Wavelength | | Embodiment 1 | | | Embodiment 2 | | | Embodiment 3 | | | Embodiment 4 | | |
| Range (nm) | | B | A | Δ | B | A | Δ | B | A | Δ | B | A | Δ |
| 425~465 | Tₘᵢₙ | 81.1 | 81.2 | 0.1 | 82.8 | 82.6 | −0.2 | 90.3 | 90.2 | −0.1 | 81.6 | 81.7 | 0.1 |
| | Tₐᵥₑ | 87.1 | 87.0 | −0.1 | 87.3 | 87.1 | −0.2 | 90.7 | 90.6 | −0.1 | 83.5 | 83.6 | 0.1 |
| 466~480 | Tₘᵢₙ | 91.1 | 91.2 | 0.1 | 89.7 | 89.6 | −0.1 | 89.3 | 89.1 | −0.2 | 87.4 | 87.3 | −0.1 |
| | Tₐᵥₑ | 91.7 | 91.8 | 0.1 | 90.7 | 90.5 | −0.2 | 89.8 | 89.7 | −0.1 | 88.5 | 88.6 | 0.1 |
| 481~560 | Tₘᵢₙ | 90.8 | 90.8 | 0.0 | 88.6 | 88.7 | 0.1 | 89.7 | 89.7 | 0.0 | 88.9 | 89 | 0.1 |
| | Tₐᵥₑ | 91.8 | 91.9 | 0.1 | 90.5 | 90.6 | 0.1 | 91.4 | 91.3 | −0.1 | 90.1 | 90.1 | 0.0 |
| 561~630 | Tₘᵢₙ | 64.9 | 64.7 | −0.3 | 67.2 | 67.3 | 0.1 | 90.0 | 89.9 | −0.1 | 76.6 | 76.8 | 0.3 |
| | Tₐᵥₑ | 82.8 | 82.6 | −0.2 | 80.9 | 67.2 | −16.9 | 91.2 | 91.1 | −0.1 | 84.7 | 84.7 | 0.0 |
| λₘₐₓ | T | 0.1 | 0.1 | 0.0 | 0.2 | 0.2 | 0.0 | 17.7 | 17.8 | 0.6 | 0.6 | 0.6 | 0.0 |

TABLE 3

| Wavelength | | | | | | | | | | | | | |
|---|---|---|---|---|---|---|---|---|---|---|---|---|---|
| (nm) or | | Comparative | | | Comparative | | | Comparative | | | Comparative | | |
| Wavelength | | Example 1 | | | Example 2 | | | Example 3 | | | Example 4 | | |
| Range (nm) | | B | A | Δ | B | A | Δ | B | A | Δ | B | A | Δ |
| 425~465 | Tₘᵢₙ | 81.2 | 86.7 | 6.3 | 82.7 | 83.6 | 1.1 | 90.4 | 89.6 | −0.9 | 81.7 | 81.0 | −0.9 |
| | Tₐᵥₑ | 87.0 | 89.5 | 2.9 | 87.2 | 86.7 | −0.6 | 90.8 | 90.2 | −0.7 | 83.6 | 83.2 | −0.5 |
| 466~480 | Tₘᵢₙ | 91.1 | 91.1 | 0.0 | 87.6 | 88.7 | 1.3 | 89.5 | 89.1 | −0.4 | 86.6 | 86.6 | 0.0 |
| | Tₐᵥₑ | 91.6 | 91.5 | −0.1 | 90.5 | 89.3 | −1.3 | 89.9 | 89.3 | −0.7 | 88.6 | 87.5 | −1.2 |
| 481~560 | Tₘᵢₙ | 90.9 | 91.2 | 0.3 | 88.7 | 84.9 | −4.3 | 89.8 | 89.4 | −0.4 | 88.8 | 86.8 | −2.3 |
| | Tₐᵥₑ | 91.8 | 91.6 | −0.2 | 90.5 | 88.3 | −2.4 | 91.5 | 90.8 | −0.8 | 90.2 | 88.5 | −1.9 |
| 561~630 | Tₘᵢₙ | 64.9 | 80.8 | 24.5 | 67.1 | 73.0 | 8.8 | 89.9 | 89.7 | −0.2 | 76.5 | 74.4 | −2.7 |
| | Tₐᵥₑ | 82.9 | 88.1 | 6.3 | 80.8 | 89.9 | −1.1 | 91.2 | 90.7 | −0.5 | 84.8 | 81.1 | −4.4 |
| λₘₐₓ | T | 0.1 | 5.2 | 5100 | 0.2 | 2.4 | 1100 | 17.6 | 29.7 | 68.8 | 0.6 | 2.3 | 283.3 |

### Test Example 2

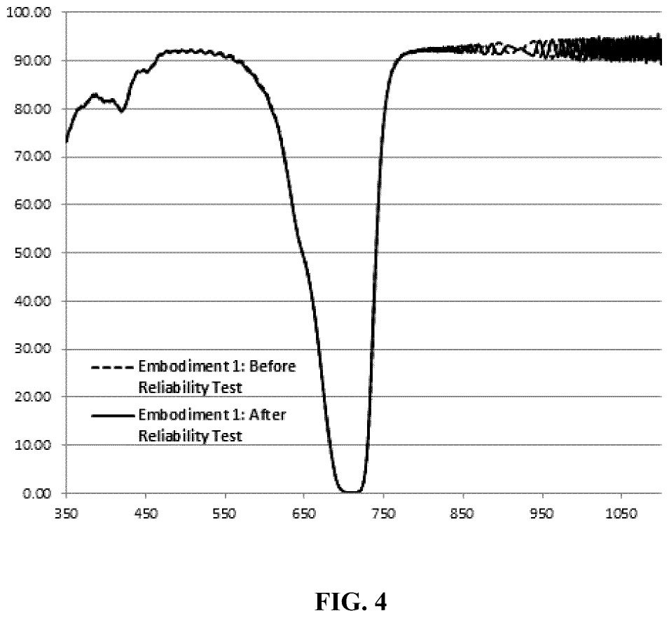

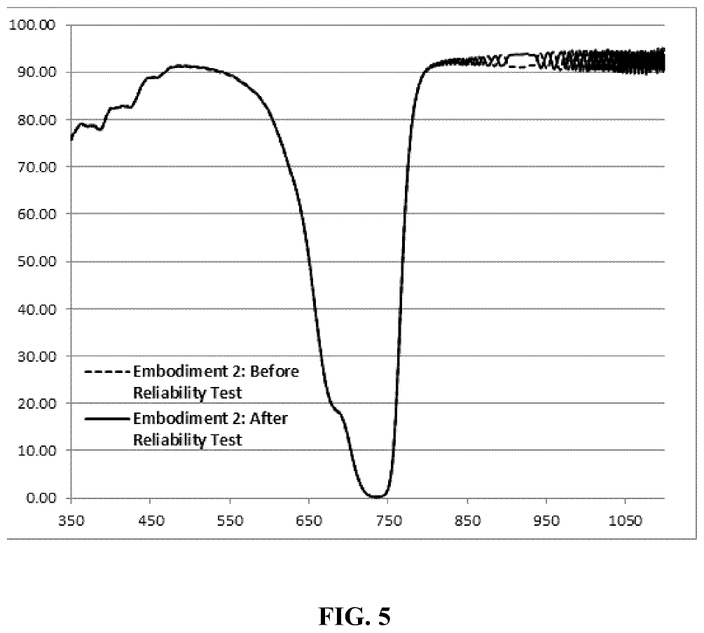

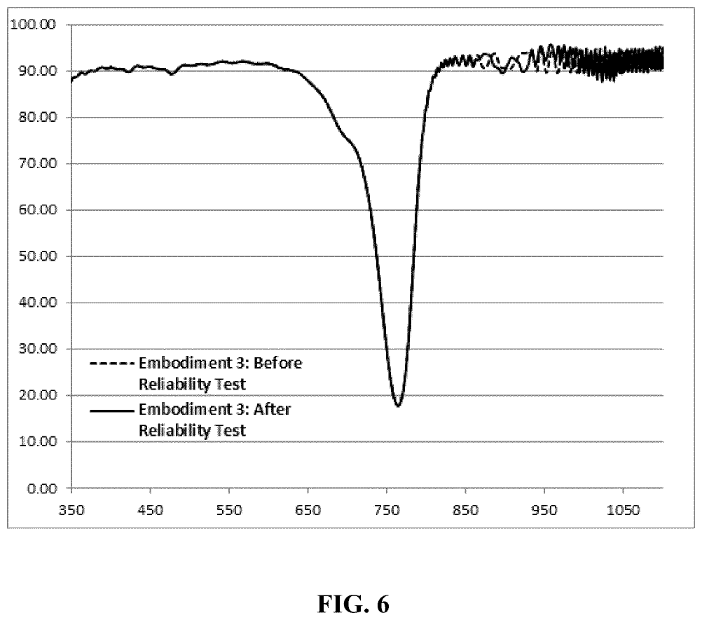

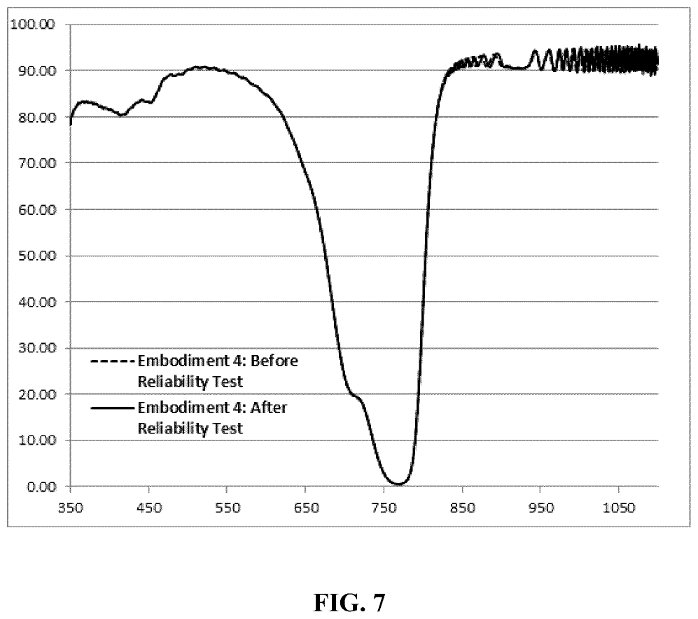

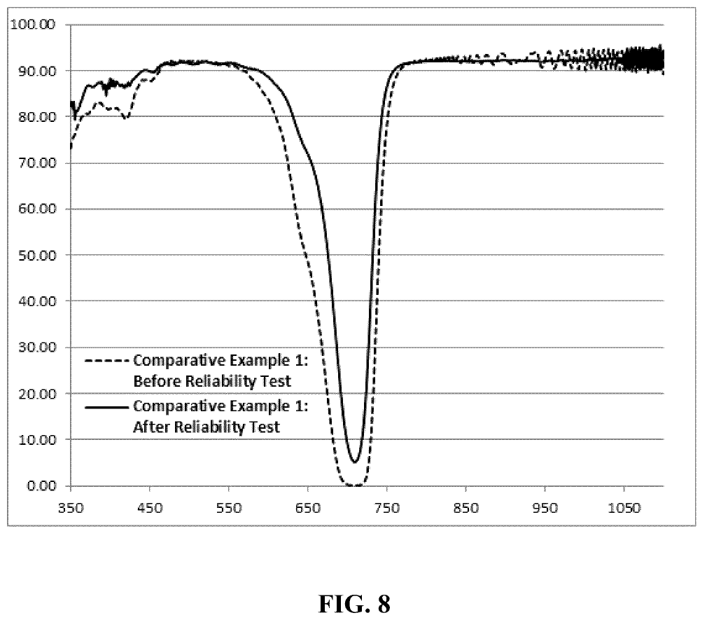

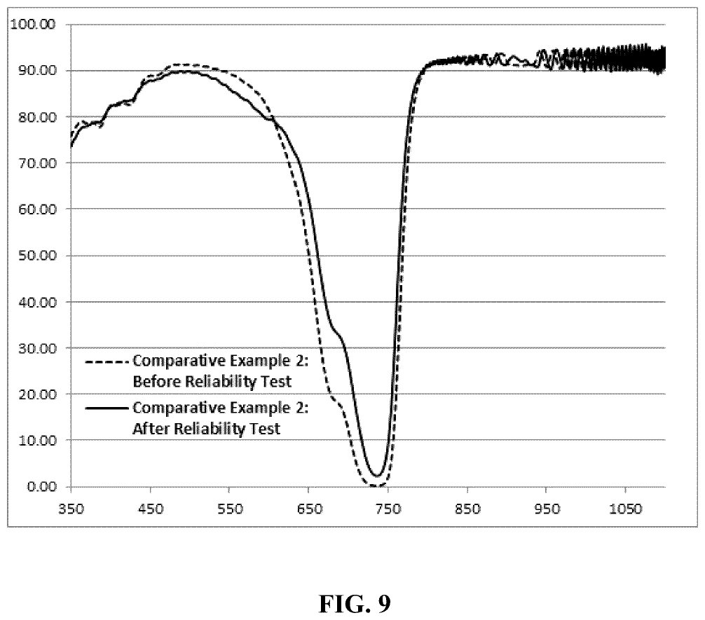

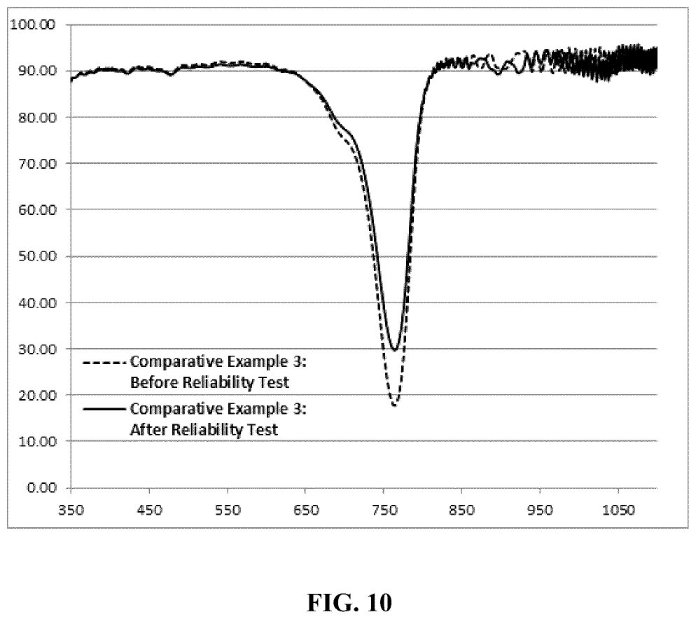

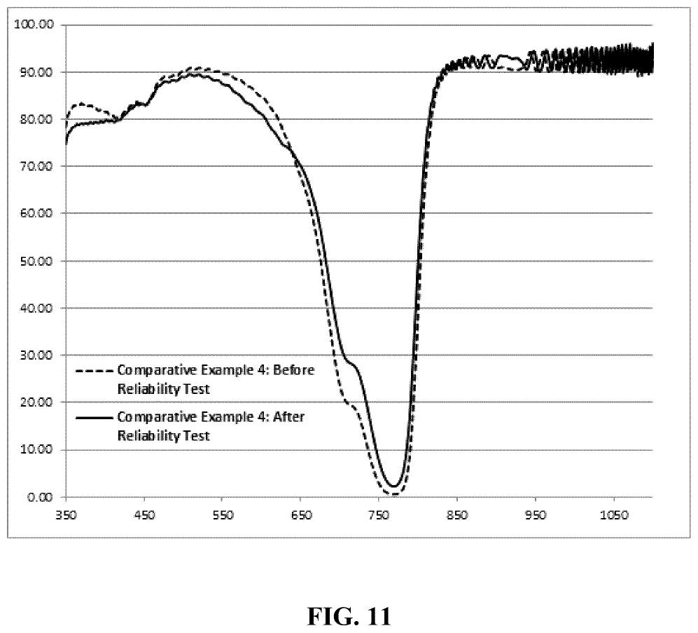

FIG. 4 to 11 show the test results for resin films manufactured by the method of Test Example 1 using the chemical compounds of Embodiments 1 to 4 (FIG. 4 to 7) and Comparative Examples 1 to 4 (FIG. 8 to 11), respectively. In FIG. 4 to 11, the horizontal axis represents wavelength (nm) and the vertical axis represents transmittance (%). In addition, the results indicated as “Before Reliability Test (a Dotted Line)” are the results immediately after manufacturing the resin film. The results indicated as “After Reliability Test (a Solid Line)” are 10 the results after performing a reliability test (same as the conditions of Test Example 1) on the resin film.

Referring to FIG. 4 to 7, the resin film using the chemical compound of Embodiment shows a small change in absorption characteristics before and after reliability test, thus, the characteristics show that the dotted line and the solid line are overlapped in FIG. 4 to 7, respectively. In contrast, the resin film using the chemical compound of Comparative Example shows in FIGS. 8 to 11, respectively, that a very large change in absorption characteristics before and after reliability test, and thus, it can be confirmed that the absorption characteristics are almost lost.

The main contents from the spectrums of FIG. 4 to 11 are summarized in Table 4 below. In Table 4, A_{f} is the transmittance at the maximum absorption wavelength of the resin film maintained at 85° C. and a relative humidity of 85% for 120 hours and λ_{f} is the maximum absorption wavelength at this time. Aᵢ is the transmittance at the maximum absorption wavelength of the resin film before being maintained at 85° C. and a relative humidity of 85% for 120 hours and λᵢ is the maximum absorption wavelength at this time.

In Table 4, ΔA is a value calculated as 100×(A_{f}−Aᵢ)/A; and Δλ is a value calculated as 100×(λ_{f}−λᵢ)/λᵢ.

TABLE 4

| | Embodiment | Embodiment | Embodiment | Embodiment | Comparative | Comparative | Comparative | Comparative |
|---|---|---|---|---|---|---|---|---|
| | 1 | 2 | 3 | 4 | Example 1 | Example 2 | Example 3 | Example 4 |
| Aᵢ | 0.1% | 0.2% | 17.7% | 0.6% | 0.1% | 0.2% | 17.6% | 0.6% |
| A_{f} | 0.1% | 0.2% | 17.8% | 0.6% | 5.2% | 2.4% | 29.7% | 2.3% |
| ΔA | 0.0 | 0.0 | 0.56 | 0.0 | 5100 | 1100 | 68.75 | 293.3 |
| λᵢ | 709 nm | 736 nm | 764 nm | 769 nm | 709 nm | 736 nm | 764 nm | 769 nm |
| λ_{f} | 709 nm | 736 nm | 764 nm | 769 nm | 709 nm | 736 nm | 764 nm | 769 nm |
| Δλ | 0% | 0% | 0% | 0% | 0% | 0% | 0% | 0% |

Comparing the results in Tables 1 to 4 and FIG. 4 to 11, it can be seen that although the chemical compounds of Embodiments and the chemical compounds of Comparative Examples have similar spectral characteristics of the chemical compounds themselves, they show significant differences in absorption characteristics and/or absorption characteristics after reliability test when applied to a resin film. From this, it can be confirmed that the chemical compound can absorb light in the infrared range due to its unique structure, has excellent heat resistance and/or oxidation resistance at the same time, and can effectively form a resin film with excellent performance.

What is claimed is:

1. A chemical compound comprising a deuterium and represented by Chemical Formula 1: 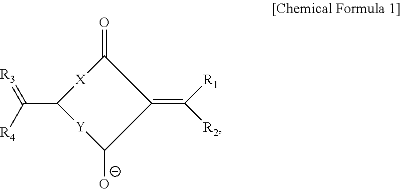
wherein, in Chemical Formula 1, one of X and Y is a single bond and the other is a double bond;
R₁ forms a structure represented by Chemical Formula 2;
R₂ is hydrogen or the deuterium or R₁ and R₂ together form a structure represented by Chemical Formula 3;
R₃ forms a structure represented by Chemical Formula 4; and
R₄ is hydrogen or the deuterium or R₃ and R₄ together form a structure represented by Chemical Formula 5 wherein, in Chemical Formula 2: 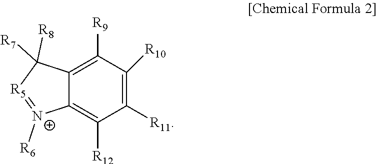
R₅ is carbon as R₁ of Chemical Formula 1;
R₆ to R₈ are each independently hydrogen, the deuterium, an alkyl group, an alkyloxy group, an alkylcarbonyl group, an alkoxy group, an alkylamino group, an alkylsilyl group, an amino group, a nitro group, a nitrile group, a hydroxy group, or a cyano group; and
R₉ to R₁₂ are each independently hydrogen or the deuterium wherein R₉ and R₁₀, R₁₀ and R₁₁, or R₁₁ and R₁₂ are linked to each other to form an aromatic structure which is unsubstituted or substituted with deuterium; wherein, in Chemical Formula 3: 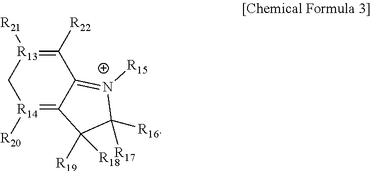
one of R₁₃ and R₁₄ is R₁ of Chemical Formula 1 and the other is R₂ of Chemical Formula 1 wherein each of R₁₃ and R₁₄ is carbon; and R₁₅ to R₂₀ are each independently hydrogen, the deuterium, an alkyl group, an alkyloxy group, an alkylcarbonyl group, an alkoxy group, an alkylamino group, an alkylsilyl group, an amino group, a nitro group, a nitrile group, a hydroxy group, a cyano group, or —NR^{a}R^{b} wherein Rₐ and R_{b} are each independently hydrogen, the deuterium, an alkyl group, an alkyloxy group, an alkylcarbonyl group, an alkoxy group, an alkylamino group, an alkylsilyl group, an amino group, a nitro group, a nitrile group, a hydroxy group or a cyano group; and
R₂₁ and R₂₂ are each independently hydrogen or the deuterium, or are linked to each other to form an aromatic structure which is unsubstituted or substituted with deuterium; wherein, in Chemical Formula 4: 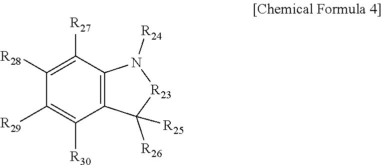
R₂₃ is carbon as R₃ of Chemical Formula 1 and R₂₄ to R₂₆ are each independently hydrogen, the deuterium, an alkyl group, an alkyloxy group, an alkylcarbonyl group, an alkoxy group, an alkylamino group, an alkylsilyl group, an amino group, a nitro group, a nitrile group, a hydroxy group, or a cyano group; and
R₂₇ to R₃₀ are each independently hydrogen or the deuterium, and R₂₇ and R₂₈, R₂₈ and R₂₉, or R₂₉ and R₃₀ are linked to each other to form a benzene structure which is unsubstituted or substituted with the deuterium; and wherein, in Chemical Formula 5: 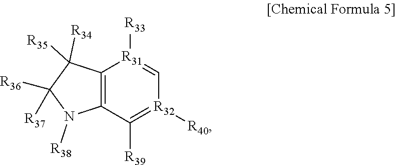
one of R₃₁ and R₃₂ is R₃ of Chemical Formula 1, and the other is R₄ of Chemical Formula 1 wherein each of R₃₁ and R₃₂ is carbon; and R₃₃ to R₃₈ are each independently hydrogen, the deuterium, an alkyl group, an alkyloxy group, an alkylcarbonyl group, an alkoxy group, an alkylamino group, an alkylsilyl group, an amino group, a nitro group, a nitrile group, a hydroxy group, a cyano group, or —NR^{a}R^{b} wherein Rₐ and R_{b} are each independently hydrogen, the deuterium, an alkyl group, an alkyloxy group, an alkylcarbonyl group, an alkoxy group, an alkylamino group, an alkylsilyl group, an amino group, a nitro group, a nitrile group, a hydroxy group, or a cyano group; and
R₃₉ and R₄₀ are each independently be hydrogen or the deuterium or are linked to each other to form a benzene structure which is unsubstituted or substituted with the deuterium.

2. The chemical compound of claim 1, wherein R₁ forms a structure of Chemical Formula 2 and R₂ is the deuterium.

3. The chemical compound of claim 2, wherein R₆ to R₈ are each independently hydrogen, the deuterium, an alkyl group, an alkyloxy group, or an alkoxy group; and R₉ to R₁₂ are each independently the deuterium in Chemical Formula 2.

4. The chemical compound of claim 2, wherein R₆ to R₈ are each independently hydrogen, the deuterium, an alkyl group, an alkyloxy group, or an alkoxy group; R₉ and R₁₀ are connected to each other to form an aromatic structure substituted with deuterium; and R₁ and R₁₂ are the deuterium in Chemical Formula 2.

5. The chemical compound of claim 2, wherein R₃ forms a structure of Chemical Formula 4;
and R₄ is the deuterium in Chemical Formula 1.

6. The chemical compound of claim 5, wherein R₂₄ to R₂₆ are each independently hydrogen, the deuterium, an alkyl group, an alkyloxy group, or an alkoxy group; and R₂₇ to R₃₀ are each deuterium in Chemical Formula 4.

7. The chemical compound of claim 5, wherein R₂₄ to R₂₆ are each independently hydrogen, the deuterium, an alkyl group, an alkyloxy group, or an alkoxy group; R₂₇ and R₂₈ are each deuterium; and R₂₉ and R₃₀ are connected to each other to form an aromatic structure substituted with the deuterium in Chemical Formula 4.

8. The chemical compound of claim 1, wherein R₁ and R₂ of Chemical Formula 1 form the structure of Chemical Formula 3 in Chemical Formula 1.

9. The chemical compound of claim 8, wherein R₁₅ to R₂₀ are each independently hydrogen, deuterium, an alkyl group, an alkyloxy group, an alkoxy group, or —NR^{a}R^{b} wherein Rₐ and Rb are each independently hydrogen, the deuterium, an alkyl group, an alkyloxy group, or an alkylcarbonyl group; and R₂₁ and R₂₂ are each deuterium in Chemical Formula 3.

10. The chemical compound of claim 8, wherein R₁₅ to R₂₀ are each independently hydrogen, the deuterium, an alkyl group, an alkyloxy group or an alkoxy group; and R₂₁ and R₂₂ are connected to each other to form an aromatic structure substituted with the deuterium in Chemical Formula 3.

11. The chemical compound of claim 8, wherein R₃ and R₄ form the structure of Chemical Formula 5 in Chemical Formula 1.

12. The chemical compound of claim 11, R₃₃ to R₃₈ are each independently hydrogen, the deuterium, an alkyl group, an alkyloxy group, an alkoxy group, or —NR^{a}R^{b} wherein Rₐ and Rb are each independently hydrogen, the deuterium, an alkyl group, an alkyloxy group, or an alkylcarbonyl group; and R₃₉ and R₄₀ are each deuterium in Chemical Formula 5.

13. The chemical compound of claim 11, wherein R₃₃ to R₃₈ are each independently hydrogen, the deuterium, an alkyl group, an alkyloxy group, or an alkoxy group; and R₃₉ and R₄₀ are connected to each other to form an aromatic structure substituted with the deuterium in Chemical Formula 5.

14. The chemical compound of claim 1, wherein a chemical compound of Chemical Formula 1 is represented by any one of Chemical Formulas 6 to 9: 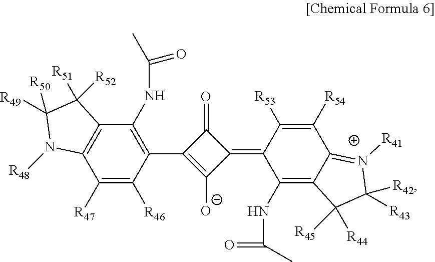
wherein, in Chemical Formula 6, R₄₁ to R₅₄ are each independently hydrogen, the deuterium, an alkyl group, an alkyloxy group, an alkylcarbonyl group, an alkoxy group, an alkylamino group, an alkylsilyl group, an amino group, a nitro group, a nitrile group, a hydroxy group, a cyano group, or —NR^{a}R^{b} wherein R^{a} and R^{b} are each independently hydrogen, the deuterium, an alkyl group, an alkyloxy group, an alkylcarbonyl group, an alkoxy group, an alkylamino group, an alkylsilyl group, an amino group, a nitro group, a nitrile group, a hydroxy group, or a cyano group;
in Chemical Formula 7: 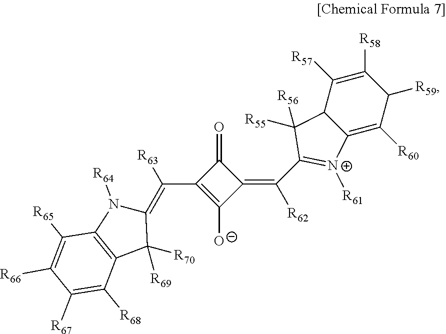
R₅₅ to R₇₀ are each independently hydrogen, the deuterium, an alkyl group, an alkyloxy group, an alkylcarbonyl group, an alkoxy group, an alkylamino group, an alkylsilyl group, an amino group, a nitro group, a nitrile group, a hydroxy group, a cyano group, or —NR^{a}R^{b} wherein R^{a} and R^{b} are each independently hydrogen, the deuterium, an alkyl group, an alkyloxy group, an alkylcarbonyl group, an alkoxy group, an alkylamino group, an alkylsilyl group, an amino group, a nitro group, a nitrile group, a hydroxy group, or a cyano group;
in Chemical Formula 8: 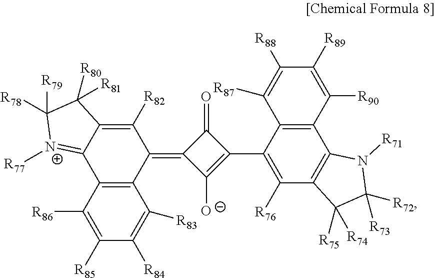
R₇₁ to R₉₀ are each independently hydrogen, the deuterium, an alkyl group, an alkyloxy group, an alkylcarbonyl group, an alkoxy group, an alkylamino group, an alkylsilyl group, an amino group, a nitro group, a nitrile group, a hydroxy group, a cyano group, or —NR^{a}R^{b} wherein R^{a} and R^{b} are each independently hydrogen, the deuterium, an alkyl group, an alkyloxy group, an alkylcarbonyl group, an alkoxy group, an alkylamino group, an alkylsilyl group, an amino group, a nitro group, a nitrile group, a hydroxy group, or a cyano group; and
in Chemical Formula 9: 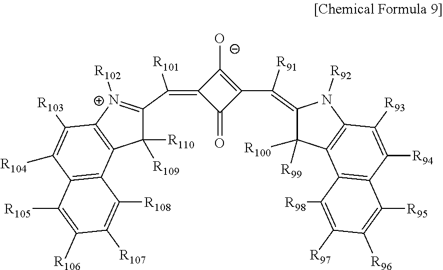
R₉₁ to R₁₁₀ are each independently hydrogen, the deuterium, an alkyl group, an alkyloxy group, an alkylcarbonyl group, an alkoxy group, an alkylamino group, an alkylsilyl group, an amino group, a nitro group, a nitrile group, a hydroxy group, a cyano group, or —NR^{a}R^{b} wherein R^{a} and R^{b} are each independently hydrogen, the deuterium, an alkyl group, an alkyloxy group, an alkylcarbonyl group, an alkoxy group, an alkylamino group, an alkylsilyl group, an amino group, a nitro group, a nitrile group, a hydroxy group, or a cyano group.

15. The chemical compound of claim 1, wherein a deuterium substitution rate is 10% or higher.

16. A composition comprising a resin component and the chemical compound of claim 1 wherein the composition contains 0.001 to 10 parts by weight of the chemical compound with respect to 100 parts by weight of the resin component.

17. A resin film comprising a resin component; and the chemical compound of claim 1 wherein the resin film exhibits an absorption maximum within a wavelength range of 650 nm to 900 nm.

18. The resin film of claim 17, wherein ΔA of Equation 1 for the resin film is 50% or less:
$$△A = 100 \times ( {A}_{f} - {A}_{i} ) / {A}_{i} , \qquad [ Equation 1 ]$$
wherein A_{f} is a transmittance at a maximum absorption wavelength of the resin film maintained at 85° C. and a relative humidity of 85% for 120 hours and Aᵢ is a transmittance at a maximum absorption wavelength of the resin film before being maintained at 85° C. and a relative humidity of 85% for 120 hours.

19. The resin film of claim 17, wherein an absolute value of Δλ of Equation 2 for the resin film is 10% or less:
$$△λ = 100 \times ( {λ}_{f} - {λ}_{i} ) / {λ}_{i} , \qquad [ Equation 2 ]$$
wherein λ_{f} is a maximum absorption wavelength of the resin film maintained at 85° C. and a relative humidity of 85% for 120 hours and λᵢ is a maximum absorption wavelength of the resin film before being maintained at 85° C. and a relative humidity of 85% for 120 hours.

20. An optical filter comprising:
a substrate; and
a resin film of claim 17 formed on one or both sides of the substrate layer.
